(19) United States
(12) Patent Application Publication
(10) Pub. No.: US 2025/0027954 A1
(43) Pub. Date: Jan. 23, 2025

(54) METHODS OF DETECTING AND TREATING CEREBRAL ANEURYSMS

(71) Applicants: UNIVERSITY OF PITTSBURGH-OF THE COMMONWEALTH SYSTEM OF HIGHER EDUCATION, Pittsburgh, PA (US); UPMC, Pittsburgh, PA (US)

(72) Inventors: Robert Max Friedlander, Pittsburgh, PA (US); Ali A. Alattar, Pittsburgh, PA (US); Kamil W. Nowicki, Pittsburgh, PA (US); Michael M. McDowell, Pittsburgh, PA (US); Aditya M. Mittal, Pittsburgh, PA (US)

(73) Assignees: UNIVERSITY PF PITTSBURGH - OF THE COMMONWEALTH SYSTEM OF HIGHER EDUCTION, Pittsburgh, PA (US); UPMC, Pittsburgh, PA (US)

(21) Appl. No.: 18/792,908

(22) Filed: Aug. 2, 2024

Related U.S. Application Data
(63) Continuation of application No. PCT/US23/12313, filed on Feb. 3, 2023.
(60) Provisional application No. 63/306,530, filed on Feb. 4, 2022.

(51) Int. Cl. G01N 33/68; A61K 31/145; A61K 31/4365; A61P 9/00; C07K 16/24; G16H 50/20

(52) U.S. Cl. CPC G01N 33/6863; A61K 31/145; A61K 31/4365; A61P 9/00; C07K 16/24; G01N 33/6893; G16H 50/20; G01N 2800/329; G01N 2800/52

(57) ABSTRACT

The present disclosure provides a whole blood, protein-based diagnostic test for presence of unruptured aneurysms and allow for tracking progression of unruptured, ruptured, and previously treated aneurysms to guide clinical decision making. Further, the present disclosure relates to methods of treating aneurysms.

### CROSS-REFERENCE TO RELATED APPLICATIONS

This application is a Continuation Application of International Patent Application No. PCT/US2023/012313, which claims priority to U.S. Provisional Patent Application Ser. No. 63/306,530, filed on Feb. 4, 2022, the content of each of which is hereby incorporated by reference in its entirety, and to each of which priority is claimed.

### TECHNICAL FIELD

The present disclosure relates to methods, compositions, and kits for detecting aneurysms. In certain embodiments, the present disclosure further includes treating cerebral aneurysms in a subject. The present disclosure also relates to biomarkers for predicting and monitoring a subject's response to a treatment.

### BACKGROUND OF THE INVENTION

Cerebral aneurysms are focal dilations of cerebral arteries that are present in 2-5% of the general population and disproportionately affect women. Almost 95% of these vascular lesions are sporadic while the remaining 5% can be attributed to familial, infectious, or traumatic causes. These lesions can rupture resulting in devastating subarachnoid hemorrhage leading to near 50% mortality and morbidity. One third of patients with subarachnoid hemorrhage have multiple aneurysms. About 20-40% of coiled aneurysms eventually recanalize requiring further surgery. Current research suggests that aneurysms form through a complex interaction of hemodynamic and inflammatory factors. Patients with aneurysms within the general population have aneurysms at vastly different stages of their disease process. Current and past efforts have failed to properly stratify patient samples at different stages of this inflammatory disease resulting in mixed samples and study results. Furthermore, most recent efforts have focused on RNA transcriptome, but RNA expression does not correlate with protein expression.

Cerebral aneurysms are typically discovered incidentally on advanced cranial imaging or when patients present with rupture. Development of medical therapies is impeded by lack of knowledge of aneurysm formation and the inability to detect them during early formation process. There currently is no blood test available to detect cerebral aneurysms in patients.

### SUMMARY OF THE INVENTION

The present disclosure relates to methods, compositions, and kits for treating and detecting aneurysms including cerebral aneurysms.

In certain embodiments, the present disclosure provides a method of treating an aneurysm in a subject in need thereof. In certain embodiments, the method comprises measuring, in a biological sample of the subject, an expression level of I-309, IL-16, IL-1 alpha, MCP-2, MIP-1 delta, and/or uPAR. In certain embodiments, the method comprises identifying the subject as having an aneurysm if the expression level of I-309, IL-16, IL-1 alpha, MCP-2, MIP-1 delta, and/or uPAR is increased relative to a first reference sample. In certain embodiments, the method comprises measuring, in a biological sample of the subject, an expression level of I-309, IL-16, MCP-4, MIP-1 delta, CXCL7/NAP-2 and/or uPAR. In certain embodiments, the method comprises identifying the subject as having an aneurysm if the expression level of I-309, IL-16, MCP-4, MIP-1 delta, CXCL7/NAP-2 and/or uPAR is increased relative to a first reference sample.

In certain embodiments, the method comprises measuring, in a biological sample of the subject, an expression level of FasL and/or CCL22. In certain embodiments, the method comprises identifying the subject as having an aneurysm if the expression level of FasL and/or CCL22 is increased relative to a first reference sample. In certain embodiments, the method comprises administering an effective amount of an aneurysm inhibitor to the subject.

In certain embodiments, the aneurysm inhibitor is a platelet inhibitor. In certain embodiments, the platelet inhibitor is selected from the group consisting of a glycoprotein IIB/IIIA inhibitor, a CXCL7 inhibitor, a CXCR1/2 inhibitor, and a combination thereof. In certain embodiments, the glycoprotein IIB/IIIA inhibitor is clopidogrel, a salt thereof, or a derivative thereof. In certain embodiments, the CXCL7 inhibitor is an antibody anti-CXCL7. In certain embodiments, the CXCR1/2 inhibitor is reparixin, a salt thereof, or a derivative thereof. In certain embodiments, the method further comprises administering a therapeutically effective amount of a secondary aneurysm inhibitor.

In certain embodiments, the present disclosure provides a method for preventing or reducing the risk of growth and/or rupture of an aneurysm in a subject in need thereof. In certain embodiments, the method comprises measuring, in a biological sample of the subject, an expression level of I-309, IL-16, IL-1 alpha, MCP-2, MIP-1 delta, and/or uPAR. In certain embodiments, the method comprises identifying the subject as having an aneurysm if the expression level of I-309, IL-16, IL-1 alpha, MCP-2, MIP-1 delta, and/or uPAR is increased relative to a first reference sample. In certain embodiments, the method comprises measuring, in a biological sample of the subject, an expression level of I-309, IL-16, MCP-4, MIP-1 delta, CXCL7/NAP-2 and/or uPAR. In certain embodiments, the method comprises identifying the subject as having an aneurysm if the expression level of I-309, IL-16, MCP-4, MIP-1 delta, CXCL7/NAP-2 and/or uPAR is increased relative to a first reference sample. In certain embodiments, the method comprises measuring, in a biological sample of the subject, an expression level of FasL and/or CCL22. In certain embodiments, the method comprises identifying the subject as having an aneurysm if the expression level of FasL and/or CCL22 is increased relative to a first reference sample. In certain embodiments, the method comprises measuring, in the biological sample, an expression level of one or more cytokines. In certain embodiments, the method comprises determining whether the subject has or is at risk of rupture of the aneurysm. In certain embodiments, the method comprises administering a therapeutically effective amount of an aneurysm inhibitor to the subject.

In certain embodiments, the aneurysm inhibitor is a platelet inhibitor. In certain embodiments, the platelet inhibitor is selected from the group consisting of a glycoprotein IIB/IIIA inhibitor, a CXCL7 inhibitor, a CXCR1/2 inhibitor, and a combination thereof. In certain embodiments, the glycoprotein IIB/IIIA inhibitor is clopidogrel, a salt thereof, or a derivative thereof. In certain embodiments, the CXCL7 inhibitor is an antibody anti-CXCL7. In certain embodiments, wherein the CXCR1/2 inhibitor is reparixin, a salt thereof, or a derivative thereof. In certain embodiments, the method further comprises administering a therapeutically effective amount of a secondary aneurysm inhibitor.

In certain embodiments, the one or more cytokines are selected from the group consisting of RANTES, IL-12 p40/p70, MIP-1α, sTNF.RI, MCP-1, MCP-2, MCP-3, MIG, IL-1ra, IL1-α, or a combination thereof. In certain embodiments, a reduced expression level of RANTES relative to a second reference sample indicates that the subject has or is at risk of rupture of the aneurysm. In certain embodiments, a reduced expression level of IL-12 p40/p70 relative to the second reference sample indicates that the subject has or is at risk of rupture of the aneurysm. In certain embodiments, a reduced expression level of MIP-1α relative to the second reference sample indicates that the subject has or is at risk of rupture of the aneurysm. In certain embodiments, a reduced expression level of sTNF.RI relative to the second reference sample indicates that the subject has or is at risk of rupture of the aneurysm. In certain embodiments, an increased expression level of MCP-1 relative to the second reference sample indicates that the subject has or is at risk of rupture of the aneurysm. In certain embodiments, an increased expression level of MCP-2 relative to the second reference sample indicates that the subject has or is at risk of rupture of the aneurysm. In certain embodiments, an increased expression level of MCP-3 relative to the second reference sample indicates that the subject has or is at risk of rupture of the aneurysm. In certain embodiments, an increased expression level of MIG relative to the second reference sample indicates that the subject has or is at risk of rupture of the aneurysm. In certain embodiments, an increased expression level of IL-1ra relative to the second reference sample indicates that the subject has or is at risk of rupture of the aneurysm. In certain embodiments, an increased expression level of IL1-α relative to the second reference sample indicates that the subject has or is at risk of rupture of the aneurysm. In certain embodiments, the aneurysm is a cerebral aneurysm. In certain embodiments, the biological sample is a blood sample, a serum sample, a plasma sample, or a cerebrospinal fluid sample. In certain embodiments, the biological sample is a blood sample.

In certain embodiments, the present disclosure provides a method of identifying a subject having or at risk of developing an aneurysm. In certain embodiments, the method comprises measuring, in a biological sample of the subject, an expression level of I-309, IL-16, IL-1 alpha, MCP-2, MIP-1 delta, and/or uPAR, wherein an increased expression level relative to a first reference sample indicates that the subject has or is at risk of developing an aneurysm. In certain embodiments, the method comprises measuring, in a biological sample of the subject, an expression level of I-309, IL-16, MCP-4, MIP-1 delta, CXCL7/NAP-2 and/or uPAR, wherein an increased expression level relative to a first reference sample indicates that the subject has or is at risk of developing an aneurysm. In certain embodiments, the method further comprises measuring, in the biological sample, an expression level of one or more cytokines. In certain embodiments, the one or more cytokines are selected from the group consisting of RANTES, IL-12 p40/p70, MIP-1α, sTNF.RI, MCP-1, MCP-2, MCP-3, MIG, IL-1ra, IL1-α, or a combination thereof.

In certain embodiments, the present disclosure provides a method of identifying a subject having or at risk of aneurysm rupture. In certain embodiments, the method comprises measuring, in a biological sample of the subject, an expression level of I-309, IL-16, IL-1 alpha, MCP-2, MIP-1 delta, and/or uPAR. In certain embodiments, the method comprises identifying the subject as having an aneurysm if the expression level of I-309, IL-16, IL-1 alpha, MCP-2, MIP-1 delta, and/or uPAR is increased relative to a first reference sample. In certain embodiments, the method comprises measuring, in a biological sample of the subject, an expression level of I-309, IL-16, MCP-4, MIP-1 delta, CXCL7/NAP-2 and/or uPAR. In certain embodiments, the method comprises identifying the subject as having an aneurysm if the expression level of I-309, IL-16, MCP-4, MIP-1 delta, CXCL7/NAP-2 and/or uPAR is increased relative to a first reference sample. In certain embodiments, the method comprises measuring, in a biological sample of the subject, an expression level of FasL and/or CCL22. In certain embodiments, the method comprises identifying the subject as having an aneurysm if the expression level of FasL and/or CCL22 is increased relative to a first reference sample.

In certain embodiments, the method comprises measuring, in the biological sample, an expression level of one or more cytokines. In certain embodiments, the method comprises determining whether the subject has or is at risk of rupture of the aneurysm.

In certain embodiments, the one or more cytokines are selected from the group consisting of RANTES, IL-12 p40/p70, MIP-1α, sTNF.RI, MCP-1, MCP-2, MCP-3, MIG, IL-1ra, IL1-α, or a combination thereof.

In certain embodiments, a reduced expression level of RANTES relative to a second reference sample indicates that the subject has or is at risk of rupture of the aneurysm. In certain embodiments, a reduced expression level of IL-12 p40/p70 relative to the second reference sample indicates that the subject has or is at risk of rupture of the aneurysm. In certain embodiments, a reduced expression level of MIP-1a relative to the second reference sample indicates that the subject has or is at risk of rupture of the aneurysm. In certain embodiments, a reduced expression level of sTNF.RI relative to the second reference sample indicates that the subject has or is at risk of rupture of the aneurysm. In certain embodiments, an increased expression level of MCP-1 relative to the second reference sample indicates that the subject has or is at risk of rupture of the aneurysm. In certain embodiments, an increased expression level of MCP-2 relative to the second reference sample indicates that the subject has or is at risk of rupture of the aneurysm. In certain embodiments, an increased expression level of MCP-3 relative to the second reference sample indicates that the subject has or is at risk of rupture of the aneurysm. In certain embodiments, an increased expression level of MIG relative to the second reference sample indicates that the subject has or is at risk of rupture of the aneurysm. In certain embodiments, an increased expression level of IL-1ra relative to the second reference sample indicates that the subject has or is at risk of rupture of the aneurysm. In certain embodiments, an increased expression level of IL1-α relative to the second reference sample indicates that the subject has or is at risk of rupture of the aneurysm.

In certain embodiments, the present disclosure provides a method of monitoring a subject's responsiveness to an anti-aneurysm treatment. In certain embodiments, the method comprises measuring, in the biological sample, an expression level of one or more cytokines. In certain embodiments, the method comprises determining whether the subject is responsive to the anti-aneurysm treatment. In certain embodiments, the subject is administered or has been administered with the anti-aneurysm treatment.

In certain embodiments, the one or more cytokines are selected from the group consisting of RANTES, IL-12 p40/p70, MIP-1α, sTNF.RI, MCP-1, MCP-2, MCP-3, MIG, IL-1ra, IL1-α, or a combination thereof. In certain embodiments, a reduced expression level of RANTES relative to a second reference sample indicates that the subject is not responsive to the anti-aneurysm treatment. In certain embodiments, a reduced expression level of IL-12 p40/p70 relative to the second reference sample indicates that the subject is not responsive to the anti-aneurysm treatment. In certain embodiments, a reduced expression level of MIP-1α relative to the second reference sample indicates that the subject is not responsive to the anti-aneurysm treatment. In certain embodiments, a reduced expression level of sTNF.RI relative to the second reference sample indicates that the subject is not responsive to the anti-aneurysm treatment. In certain embodiments, an increased expression level of MCP-1 relative to the second reference sample indicates that the subject is not responsive to the anti-aneurysm treatment. In certain embodiments, an increased expression level of MCP-2 relative to the second reference sample indicates that the subject is not responsive to the anti-aneurysm treatment. In certain embodiments, an increased expression level of MCP-3 relative to the second reference sample indicates that the subject is not responsive to the anti-aneurysm treatment. In certain embodiments, an increased expression level of MIG relative to the second reference sample indicates that the subject is not responsive to the anti-aneurysm treatment. In certain embodiments, an increased expression level of IL-1ra relative to the second reference sample indicates that the subject is not responsive to the anti-aneurysm treatment. In certain embodiments, an increased expression level of IL1-α relative to the second reference sample indicates that the subject is not responsive to the anti-aneurysm treatment.

In certain embodiments, the aneurysm is a cerebral aneurysm. In certain embodiments, the biological sample is a blood sample, a serum sample, a plasma sample, or a cerebrospinal fluid sample. In certain embodiments, the biological sample is a blood sample.

In certain embodiments, the first reference sample comprises an expression level of I-309, IL-16, IL-1 alpha, MCP-2, MIP-1 delta, and/or uPAR in a population of individuals free of aneurysm. In certain embodiments, the expression level of I-309, IL-16, IL-1 alpha, MCP-2, MIP-1 delta, and/or uPAR protein is measured. In certain embodiments, the first reference sample comprises an expression level of I-309, IL-16, MCP-4, MIP-1 delta, CXCL7/NAP-2 and/or uPAR in a population of individuals free of aneurysm. In certain embodiments, the expression level of −309, IL-16, MCP-4, MIP-1 delta, CXCL7/NAP-2 and/or uPAR protein is measured.

In certain embodiments, the first reference sample comprises an expression level of FasL and/or CCL22 in a population of individuals free of aneurysm. In certain embodiments, the expression level of FasL and/or CCL22 protein is measured.

In certain embodiments, the second reference sample comprises an expression level of RANTES, IL-12 p40/p70, MIP-1α, sTNF.RI, MCP-1, MCP-2, MCP-3, MIG, IL-1ra, IL1-α, or a combination thereof in a population of individuals with unruptured aneurysm. In certain embodiments, the expression level of RANTES, IL-12 p40/p70, MIP-1α, sTNF.RI, MCP-1, MCP-2, MCP-3, MIG, IL-1ra, IL1-α protein is measured.

In certain embodiments, the present disclosure provides a method of identifying a subject having or at risk of developing an aneurysm by one or more computing systems. In certain embodiments, the computing systems can receive, from a client device via a user interface of a software executing on the client device, one or more inputs associated with the subject. The computing systems can then determine, based on one or more models, one or more measures regarding aneurysm presence and aneurysm rupture. In certain embodiments, the one or more measures can comprise one or more of a first probability of the subject harboring an aneurysm, a second probability of an ruptured aneurysm in the subject, or a third probability of an aneurysm with impending rupture in the subject. The computing systems can further send, to the client device via the user interface, instructions for presenting the one or more determined measures regarding aneurysm presence and aneurysm rupture.

In certain embodiments, one or more computer-readable non-transitory storage media embodying software is operable when executed to receive, from a client device via a user interface of a software executing on the client device, one or more inputs associated with the subject. The computer-readable non-transitory storage media embodying software is further operable when executed determine, based on one or more models, one or more measures regarding aneurysm presence and aneurysm rupture. In certain embodiments, the one or more measures can comprise one or more of a first probability of the subject harboring an aneurysm, a second probability of an ruptured aneurysm in the subject, or a third probability of an aneurysm with impending rupture in the subject. The computer-readable non-transitory storage media embodying software is further operable when executed to send, to the client device via the user interface, instructions for presenting the one or more determined measures regarding aneurysm presence and aneurysm rupture.

In certain embodiments, a system can comprise one or more processors and a non-transitory memory coupled to the processors comprising instructions executable by the processors. The processors are operable when executing the instructions to receive, from a client device via a user interface of a software executing on the client device, one or more inputs associated with the subject. The processors are further operable when executing the instructions to determine, based on one or more models, one or more measures regarding aneurysm presence and aneurysm rupture. In certain embodiments, the one or more measures can comprise one or more of a first probability of the subject harboring an aneurysm, a second probability of an ruptured aneurysm in the subject, or a third probability of an aneurysm with impending rupture in the subject. The processors are further operable when executing the instructions to send, to the client device via the user interface, instructions for presenting the one or more determined measures regarding aneurysm presence and aneurysm rupture.

Furthermore, the disclosed embodiments of the methods, computer readable non-transitory storage media, and systems can have further non-limiting features as described below.

In certain embodiments, the one or more inputs can comprise one or more of demographic information, a co-morbidity, an aneurysm size, an aneurysm location, or a cytokine. In one feature, the one or more inputs can comprise one or more cytokines selected from the group consisting of I-309, IL-16, MCP-4, MIP-1 delta, CXCL7/NAP-2 and/or uPAR, or a combination thereof. In one feature, the one or more inputs can comprise one or more cytokines selected from the group consisting of I-309, IL-16, IL-1 alpha, MCP-2, MIP-1 delta, and/or uPAR, or a combination thereof. In one feature, the one or more inputs can comprise one or more cytokines selected from FasL, CCL22, or a combination thereof. In one feature, the one or more inputs can comprise one or more cytokines selected from the group consisting of RANTES, IL-12 p40/p70, MIP-1α, sTNF.RI, MCP-1, MCP-2, MCP-3, MIG, IL-1ra, IL1-α, or a combination thereof.

In certain embodiments, the user interface can be operable for querying aneurysm records associated with a plurality of subjects. In certain embodiments, the user interface can be operable for querying the one or more measures over a particular time period.

In certain embodiments, the one or more models can be generated based on one or more of retrospective human cytokine data or sample stratification based on t-SNE inflammatory cytokine analysis. In certain embodiments, the one or models can be generated based on data collected from a plurality of subjects at one or more time periods.

### BRIEF DESCRIPTION OF THE DRAWINGS

FIG. 1 shows the current clinical paradigm for treating patients suspected of having an aneurysm.

FIG. 2 shows a new clinical approach based on the present disclosure in patients with known cerebral aneurysm(s).

FIG. 3 shows a new clinical approach based on the present disclosure in family members of patients with aneurysm(s) and high-risk patient groups.

FIGS. 4A-4D show murine cerebral aneurysm inflammation cytokine levels changed over time with treatment and detected aneurysm formation in the murine model. FIG. 4A shows a heatmap depicting cytokine expression in aneurysmal mice at various time points (2- or 3-weeks) and in mice treated with clopidogrel or reparixin. Clopidogrel had a moderate response while anti-CXCR1/2 blockade (reparixin) showed robust anti-inflammatory effects. (n=3 each). FIG. 4B shows a panel of 8 cytokines (variables), based on absolute or relative difference, was used to differentiate between 2- or 3-week aneurysms from controls. FIG. 4C shows a 100% sensitivity and specificity were used to differentiate between aneurysmal samples and reference serum. FIG. 4D shows an iterative approach used to narrow down the putative significant biomarkers from animal 8-cytokine panel to arrive at a 5-cytokine panel test applied to human samples. The most significant variables were included. Variables were excluded if they resulted in the reversal of associations (e.g. increased the odds of aneurysm formation despite being protective against aneurysm formation on the univariable analysis)

FIGS. 5A-5E show analysis of human tissue obtained from an IRB-approved de-identified human tissue bank. FIG. 5A shows a combination figure with an illustration depicting the location of aneurysms within the Circle of Willis located on the inferior surface of the brain. FIG. 5A further shows an analysis of peripheral blood samples from patients with aneurysms, with no reported unusual findings. For analysis, human aneurysm dome was paired with STA tissue, and venous blood samples (n=21), including 14 female and 7 male samples. Of the samples analyzed, 2 were ruptured and 19 unruptured aneurysms. The samples were obtained from patients including nine (9) smokers, four (4) non-smokers, and eight (8) of unknown status. Human aneurysm cytokine fingerprint profiling shows a heatmap of cytokine expression in human peripheral blood and aneurysm tissue, generated from semi-quantitative array analysis of 120 different cytokines. FIG. 5B shows an example of a preliminary two-cytokine model to predict and differentiate between aneurysmal samples and reference serum with 81.0% sensitivity and 75.0% specificity. FIG. 5C shows the application of a 6-cytokine panel test derived from the murine model (FIG. 4) and human cytokine arrays (FIG. 5A) was used to predict and differentiate between aneurysmal samples and reference serum with 95.2% sensitivity and 90.9% specificity (blinded) (peripheral blood n=21, aneurysm dome n=3, reference serum n=3, total controls=11). Model performance in training set table shows various sensitivities, specificities, PPVs, and NPVs as probability threshold values in the model are varied. This represents the most balanced model in terms of sensitivity and specificity. FIG. 5D shows the application of a 6-cytokine panel test derived from the murine model (FIG. 4) and human cytokine arrays (FIG. 5A) was used to predict and differentiate between aneurysmal samples and reference serum with 100% sensitivity and 75% specificity (blinded) (peripheral blood n=21, aneurysm dome n=3, reference serum n=3, total controls=11). Model performance in training set table shows various sensitivities, specificities, PPVs, and NPVs as probability threshold values in the model are varied. FIG. 5E shows the application of a 6-cytokine panel test derived from the murine model (FIG. 4) and human cytokine arrays (FIG. 5A) was used to predict and differentiate between ruptured aneurysmal samples and unruptured aneurysmal samples with 100% sensitivity and 100% specificity (blinded) (peripheral blood of unruptured patients n=19, peripheral blood of ruptured patients n=2). Model performance in training set table shows various sensitivities, specificities, PPVs, and NPVs as probability threshold values in the model are varied. FIG. 5E shows a two-cytokine model for predicting aneurysm presence.

FIG. 6 shows an illustration depicting the blood test approach to detect human cerebral aneurysm formation. The development of a blood test for cerebral aneurysm detection was established using a pre-clinical mouse aneurysm model and previously collected, de-identified human blood and cerebral aneurysm samples.

FIG. 7 shows a bar plot depicting cytokine expression in human aneurysms. Samples analyzed included human peripheral blood (PVB) and cerebral aneurysm dome (dome), reference control serum, meningioma patients, and metastasis patients.

FIGS. 8A and 8B show a graphic representation of principal component analysis for defining inflammatory signature sub-groups. FIG. 8A shows that a simple application of a multi-cytokine panel (in this case a panel with 120 different cytokines) does not distinguish peripheral blood samples from patients with aneurysms when compared to reference controls. FIG. 8B shows subgroup analysis after application of models from FIG. 5C illustrating that the assay is able to differentiate peripheral blood samples from patients harboring aneurysm(s) from reference controls.

FIGS. 9A-9D shows inflammatory panels for predicting aneurysm sub-groups. FIG. 9A shows the application of a subsequent 6-cytokine panel test derived from the human cytokine arrays (FIG. 5A) to predict and differentiate between subgroup 2 and other groups within the peripheral blood of patients harboring aneurysms (n=21). Model performance in training set table shows various sensitivities, specificities, PPVs, and NPVs as probability threshold values in the model are varied. FIG. 9B shows the application of a subsequent 4-cytokine panel test derived from the human cytokine arrays (FIG. 5A) to predict and differentiate between subgroup 3 and other groups within the peripheral blood of patients harboring aneurysms (n=21). Model performance in training set table shows various sensitivities, specificities, PPVs, and NPVs as probability threshold values in the model are varied. FIG. 9C shows the application of a subsequent 3-cytokine panel test derived from the human cytokine arrays (FIG. 5A) to predict and differentiate between subgroup 4 and other groups within the peripheral blood of patients harboring aneurysms (n=21). Model performance in training set table shows various sensitivities, specificities, PPVs, and NPVs as probability threshold values in the model are varied. FIG. 9D shows the application of a subsequent 4-cytokine panel test derived from the human cytokine arrays (FIG. 5A) to predict and differentiate between subgroup 5 and other groups within the peripheral blood of patients harboring aneurysms (n=21). Model performance in training set table shows various sensitivities, specificities, PPVs, and NPVs as probability threshold values in the model are varied.

FIGS. 10A and 10B show the process of developing a diagnostic test for aneurysm formation and rupture risk. FIG. 10A shows representative images depicting a sample data set obtained from a cytokine array panel. A cytokine signature profile was generated and was used to evaluate patient status. FIG. 10B shows a graphic summarizing the steps taken to obtain a final model for human cerebral aneurysm detection and rupture risk. For diagnostic tests developed in FIGS. 5C, 5D, and 9A-9D, the least absolute shrinkage and selection operator method (LASSO method) was used. For diagnostic test developed in FIGS. 5A and 5E, t-test and logistic regression was used.

FIG. 11 shows a simplified schematic summarizing the application of a cytokine panel to diagnose cerebral aneurysm formation and rupture.

FIGS. 12A-12F show a mobile/based software for predicting the probability of aneurysm presence and the probability of aneurysm rupture for a patient. FIG. 12A shows a user interface of the software for inputting cytokines associated with the patient. FIG. 12B shows a user interface of the software for inputting the patient's age. FIG. 12C shows a user interface of the software for inputting the patient's information. FIG. 12D shows a user interface of the software for inputting the patient's additional information. FIG. 12E shows a user interface displaying the predicted probability of aneurysm presence and the probability of aneurysm rupture of the patient. FIG. 12F shows a user interface displaying the measures of risk of aneurysm and risk of rupture over time.

FIG. 13 shows an example computing system for identifying a subject having or at risk of developing an aneurysm.

FIG. 14 shows a proposed testing and treatment scheme utilizing diagnostic tests as follows: CAT-7 Panel 1—diagnostic tests developed in FIG. 5C (balanced), FIG. 5D (high-sensitivity) or FIG. 5E. CAT-7 Panel 2—diagnostic tests developed in FIGS. 9A-9D.

### DETAILED DESCRIPTION OF THE INVENTION

The present disclosure is based, in part, on the observation that the development of aneurysms (e.g., cerebral aneurysms) has a dynamic inflammatory profile that changes over time. The present disclosure shows that a forming or growing aneurysm possesses a specific profile because it experiences a dynamic inflammatory micro-environment. The present disclosure provides a whole blood, protein-based diagnostic test for presence of unruptured aneurysms and allows for tracking progression of unruptured, ruptured, and previously aneurysms to guide clinical decision making. Further, the present disclosure relates to methods of treating aneurysms.

Non-limiting embodiments of the present disclosure are described by the present specification and Examples. For purpose of clarity and not by way of limitation, the detailed description is divided into the following subsections:
1. Definitions;
2. Biomarkers and Diagnostic Methods;
3. Web-based and/or Mobile-based Software;
4. Methods of Treatment; and
5. Kits.

### 1. Definitions

The terms used in this specification generally have their ordinary meanings in the art, within the context of this disclosure and in the specific context where each term is used. Certain terms are discussed below, or elsewhere in the specification, to provide additional guidance to the practitioner in describing the compositions and methods of the disclosure and how to make and use them.

As used herein, the use of the word “a” or “an” when used in conjunction with the term “comprising” in the claims and/or the specification may mean “one,” but it is also consistent with the meaning of “one or more,” “at least one,” and “one or more than one.” Still further, the terms “having,” “including,” “containing” and “comprising” are interchangeable and one of skill in the art is cognizant that these terms are open-ended terms.

The terms “comprise(s),” “include(s),” “having,” “has,” “can,” “contain(s),” and variants thereof, as used herein, are intended to be open-ended transitional phrases, terms, or words that do not preclude the possibility of additional acts or structures. The present disclosure also contemplates other embodiments “comprising,” “consisting of”, and “consisting essentially of,” the embodiments or elements presented herein, whether explicitly set forth or not.

The term “about” or “approximately” means within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, preferably up to 10%, more preferably up to 5%, and more preferably still up to 1% of a given value. Alternatively, particularly with respect to biological systems or processes, the term can mean within an order of magnitude, preferably within 5-fold, and more preferably within 2-fold, of a value.

An “individual” or “subject” herein is a vertebrate, such as a human or non-human animal, for example, a mammal. Mammals include, but are not limited to, humans, non-human primates, farm animals, sport animals, rodents, and pets. Non-limiting examples of non-human animal subjects include rodents such as mice, rats, hamsters, and guinea pigs; rabbits; dogs; cats; sheep; pigs; goats; cattle; horses; and non-human primates such as apes and monkeys.

As used herein, the term “disease” refers to any condition or disorder that damages or interferes with the normal function of a cell, tissue, or organ.

An “effective amount” or “therapeutically effective amount” is an amount effective, at dosages and for periods of time necessary, that produces a desired effect, e.g., the desired therapeutic or prophylactic result. In certain embodiments, an effective amount can be formulated and/or administered in a single dose. In certain embodiments, an effective amount can be formulated and/or administered in a plurality of doses, for example, as part of a dosing regimen.

As used herein, the term “derivative” refers to a chemical compound with a similar core structure. For example, trichloromethane (chloroform) is a derivative of methane.

The term “enantiomers” refers to a pair of stereoisomers that are non-superimposable mirror images of each other. A 1:1 mixture of a pair of enantiomers is a “racemic” mixture or a racemate. The term is used to designate a racemic mixture where appropriate.

The term “enantiopure” refers to a sample that within the limits of detection consists of a single enantiomer.

The term “diastereoisomers” refers to stereoisomers that have at least two asymmetric atoms, but which are not mirror-images of each other. The absolute stereochemistry is specified according to the Cahn-Ingold-Prelog R S system. When a compound is a pure enantiomer, the stereochemistry at each chiral carbon can be specified by either R or S. Resolved compounds whose absolute configuration is unknown can be designated (+) or (−) depending on the direction (dextro or levorotatory) in which they rotate plane polarized light at the wavelength of the sodium D line.

The term “isomers” refers to different compounds that have the same molecular formula but differ in arrangement and configuration of the atoms. Also, as used herein, the term “stereoisomer” refers to any of the various stereo isomeric configurations which can exist for a given compound of the presently disclosed subject matter and includes geometric isomers. It is understood that a substituent can be attached at a chiral center of a carbon atom. Also, as used herein, the term “constitutional isomers” refers to different compounds that have the same numbers of, and types of, atoms but the atoms are connected differently.

“Inhibitors” or “antagonists,” as used herein, refer to modulating compounds that reduce, decrease, block, prevent, delay activation, inactivate, desensitize, or down-regulate the biological activity and/or expression of a receptor or pathway of interest. The term “antagonist” includes full, partial, and neutral antagonists as well as inverse agonists.

The term “nucleic acid molecule” and “nucleotide sequence,” as used herein, refers to a single or double-stranded covalently-linked sequence of nucleotides in which the 3′ and 5′ ends on each nucleotide are joined by phosphodiester bonds. The nucleic acid molecule can include deoxyribonucleotide bases or ribonucleotide bases, and can be manufactured synthetically in vitro or isolated from natural sources.

The terms “polypeptide,” “peptide,” “amino acid sequence” and “protein,” used interchangeably herein, refer to a molecule formed from the linking of at least two amino acids. The link between one amino acid residue and the next is an amide bond and is sometimes referred to as a peptide bond. A polypeptide can be obtained by a suitable method known in the art, including isolation from natural sources, expression in a recombinant expression system, chemical synthesis, or enzymatic synthesis. The terms can apply to amino acid polymers in which one or more amino acid residue is an artificial chemical mimetic of a corresponding naturally occurring amino acid, as well as to naturally occurring amino acid polymers and non-naturally occurring amino acid polymers.

As used herein, the term “aneurysm” refers to a bulging, weak area in the wall of a blood vessel. An aneurysm can occur in any blood vessel, but most often develops in an artery rather than a vein. An aneurysm can be categorized by its location, shape, and cause. For example, an aneurysm may be found in many areas of the body, such as brain (cerebral aneurysm), aorta (aortic aneurysm), neck, intestines, kidney, spleen, legs.

As used herein, the term “watchful waiting” refers to an approach of treating a medical condition that involves a period of time to wait and watch for further symptoms (e.g., signs of disease) to develop, rather than immediate treatment such as surgery or administration of medication. During this time, repeated testing can be performed.

As used herein, the term “treating” or “treatment” refers to clinical intervention in an attempt to alter the disease course of the individual or cell being treated, and can be performed either for prophylaxis or during the course of clinical pathology. Therapeutic effects of treatment include, without limitation, preventing occurrence or recurrence of disease, alleviation of symptoms, diminishment of any direct or indirect pathological consequences of the disease, preventing aneurysms, decreasing the rate of disease progression, amelioration or palliation of the disease state, and remission or improved prognosis. By preventing progression of a disease or disorder, a treatment can prevent deterioration due to a disorder in an affected or diagnosed subject or a subject suspected of having the disorder, but also a treatment may prevent the onset of the disorder or a symptom of the disorder in a subject at risk for the disorder or suspected of having the disorder.

### 2. Biomarkers and Diagnostic Methods

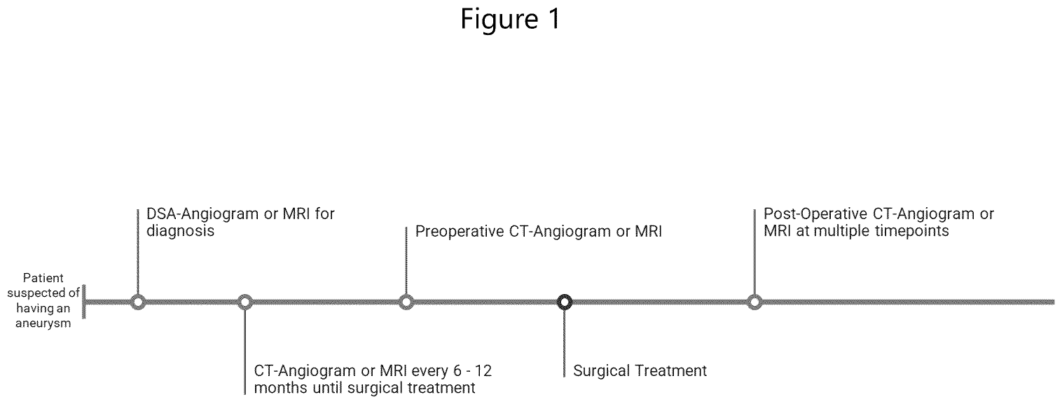

Current clinical approach for patients suspected of having an aneurysm is based on a watchful waiting approach. For the initial diagnosis, as shown in FIG. 1, imagining diagnostic tools such as DSA-angiogram or MRI can be used. Additional examinations, such as CT-angiogram or MRI, are then performed every 6 to 12 months until surgical treatment is needed. Further imagining-based analysis are then performed before and after the surgical treatment.

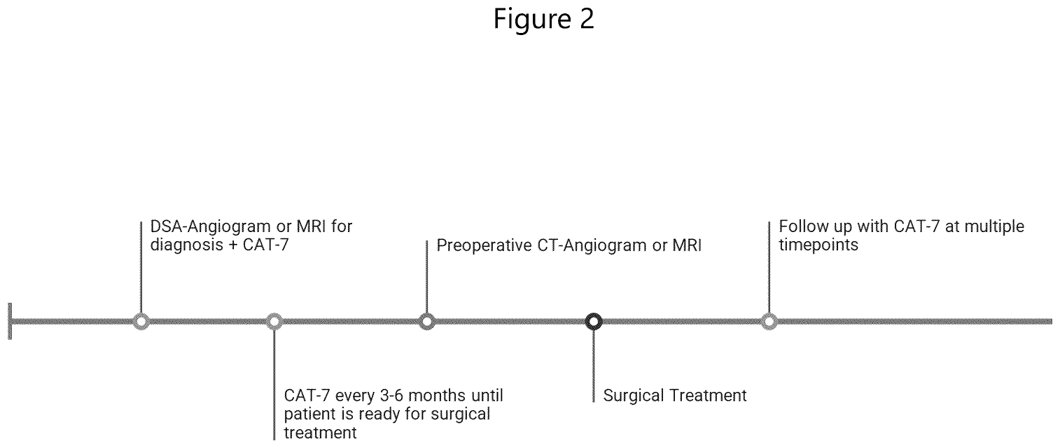

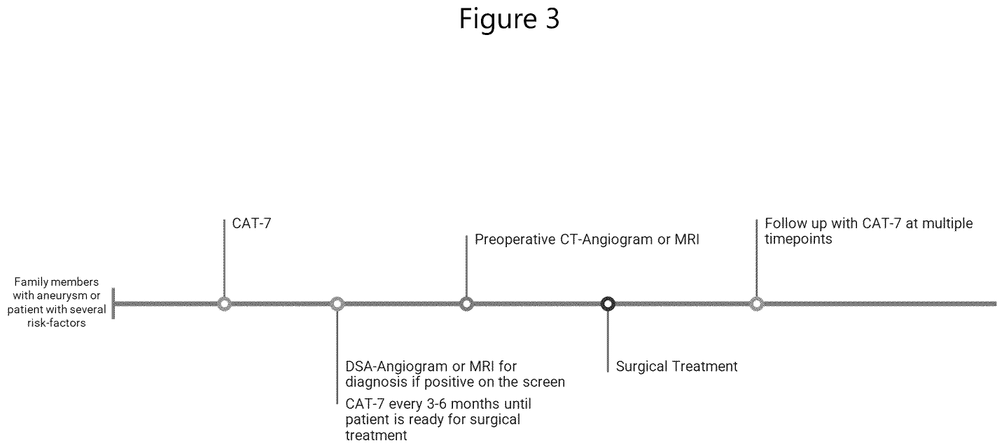

The present disclosure provides methods that can be used, with or without imaging-based examination (e.g., MRI), in a new clinical approach. FIG. 2 provides an exemplary outline of the clinical paradigm including the methods disclosed herein. Furthermore, the outlined clinical paradigm can be extended to family members of patients with aneurysm(s) and high-risk patient groups, as demonstrated in FIG. 3. Once a patient is suspected of having an aneurysm, the methods disclosed herein are performed to determine whether the patient i) has or is at risk of developing an aneurysm, and/or ii) has or is at risk of aneurysm rupture. The methods disclosed herein can be then performed every 3 to 6 months until the patient is ready for surgical treatment. Finally, the methods disclosed herein can be used in the post-operative phase (e.g., every 3 to 6 months after the surgery). It will be clear to the skilled in the art that the methods disclosed herein allow to a significant improvement of patient's clinical management and reduction of costs for analysis of the clinical status.

In certain embodiments, the present disclosure provides methods for identifying a subject having an aneurysm. In certain embodiments, the present disclosure also provides methods for identifying a subject at risk of developing an aneurysm. In certain embodiments, the methods include measuring an expression level of a Fas Ligand (FasL) biomarker. In certain embodiments, the methods include measuring an expression level of a C-C motif chemokine 22 (CCL22) biomarker. In certain embodiments, the methods include measuring an expression level of FasL and CCL22. In certain embodiments, the methods include measuring an expression level of a C-C motif chemokine ligand 1 (CCL1 or I-309) biomarker. In certain embodiments, the methods include measuring an expression level of an interleukin 16 (IL-16) biomarker. In certain embodiments, the methods include measuring an expression level of an interleukin 1 (IL-1) biomarker. In certain embodiments, the methods include measuring an expression level of an interleukin 1 alpha (IL-1 alpha) biomarker. In certain embodiments, the methods include measuring an expression level of a C-C motif chemokine ligand 8 (CCL8 or MCP-2) biomarker. In certain embodiments, the methods include measuring an expression level of a C-C motif chemokine ligand 13 (CCL13 or MCP-4) biomarker. In certain embodiments, the methods include measuring an expression level of a C-C motif chemokine ligand 15 (CCL15 or MIP-1 delta) biomarker. In certain embodiments, the methods include measuring an expression level of a urokinase plasminogen activator surface receptor (uPAR) biomarker. In certain embodiments, the methods include measuring an expression level of a C-X-C motif chemokine ligand 7 (CXCL7/NAP-2) biomarker. In certain embodiments, the methods include measuring an expression level of I-309, IL-16, IL-1 alpha, MCP-2, MIP-1 delta, and uPAR. In certain embodiments, the methods include measuring an expression level of I-309, IL-16, MCP-4, MIP-1 delta, CXCL7/NAP-2 and uPAR. In certain embodiments, the methods for identifying a subject having an aneurysm further comprises defining the aneurysms based on distinct inflammatory signature subgroup. In certain embodiments, the methods include measuring an expression level of a GDNF biomarker. In certain embodiments, the methods include measuring an expression level of a IGFB p-4 biomarker. In certain embodiments, the methods include measuring an expression level of a IL-3 biomarker. In certain embodiments, the methods include measuring an expression level of a SCF biomarker. In certain embodiments, the methods include measuring an expression level of a HGF biomarker. In certain embodiments, the methods include measuring an expression level of a IL-17 biomarker. In certain embodiments, the methods include measuring an expression level of a IL-16 biomarker. In certain embodiments, the methods include measuring an expression level of a dtk biomarker. In certain embodiments, the methods include measuring an expression level of a EGF-R biomarker. In certain embodiments, the methods include measuring an expression level of a MIP-3 beta biomarker. In certain embodiments, the methods include measuring an expression level of a GCP-2 biomarker. In certain embodiments, the methods include measuring an expression level of a LIGHT biomarker. In certain embodiments, the methods include measuring an expression level of a MCP-2 biomarker. In certain embodiments, the methods include measuring an expression level of a fractalkine biomarker. In certain embodiments, the methods include measuring an expression level of a IGFB P-1 biomarker. In certain embodiments, the methods include measuring an expression level of a bFGF biomarker. In certain embodiments, the methods include measuring an expression level of a IGF-1 SR biomarker. In certain embodiments, the methods include measuring an expression level of GDNF, IGFB p-4, IL-3, SCF, HGF, and IL17. In certain embodiments, the methods include measuring an expression level of GDNF, IGFB p-4, IL-3, SCF, HGF, IL17. In certain embodiments, the methods include measuring an expression level of GP-2, LIGHT, MCP-2. In certain embodiments, the methods include measuring an expression level of fractalkine, IGFB P-1, bFGF, IGF-I SR. In certain embodiments, an increased expression level relative to a first reference sample indicates that the subject has or is at risk of developing an aneurysm. In certain embodiments, an decreased expression level relative to a first reference sample indicates that the subject has or is at risk of developing an aneurysm.

As used herein, the term “first reference sample” refers to a control for a biomarker that is to be detected in a biological sample of a subject. For example, a control can be the level of a biomarker from a healthy individual without aneurysm. In certain embodiments, a reference sample can be the level of a biomarker detected in a healthy individual that has never had an aneurysm. In certain embodiments, a reference sample can be the level of a biomarker detected in a cohort of healthy individuals that have never had an aneurysm. In certain embodiments, the reference sample can be a predetermined level of a biomarker that indicates the presence of an aneurysm in a subject.

In certain embodiments, the methods further include measuring an expression level of one of more cytokines. In certain embodiments, the one or more cytokines are selected from the group consisting of RANTES, IL-12 p40/p70, MIP-1α, sTNF.RI, and MCP-1, MCP-2, MCP-3, or a combination thereof. In certain embodiments, the methods further include measuring an expression level of RANTES, IL-12 p40/p70, MIP-1α, sTNF.RI, MCP-1, MCP-2, MCP-3, or a combination thereof. In certain embodiments, the one or more cytokines are selected from the group consisting of MCP-2, IL12-p40, sTNF-RI, MIG, IL-1ra, IL1 alpha, or a combination thereof. In certain embodiments, the methods further include measuring an expression level of MCP-2, IL12-p40, sTNF-RI, MIG, IL-1ra, IL1 alpha, or a combination thereof.

In certain embodiments, the present disclosure provides methods for identifying a subject having an aneurysm rupture. In certain embodiments, the present disclosure also provides methods for identifying a subject at risk of aneurysm rupture. In certain embodiments, the methods include measuring an expression level of a FasL biomarker. In certain embodiments, the methods include measuring an expression level of a CCL22 biomarker. In certain embodiments, the methods include measuring an expression level of FasL and CCL22. In certain embodiments, the methods include measuring an expression level of a C-C motif chemokine ligand 1 (CCL1 or I-309) biomarker. In certain embodiments, the methods include measuring an expression level of an interleukin 16 (IL-16) biomarker. In certain embodiments, the methods include measuring an expression level of an interleukin 1 (IL-1) biomarker. In certain embodiments, the methods include measuring an expression level of an interleukin 1 alpha (IL-1 alpha) biomarker. In certain embodiments, the methods include measuring an expression level of a C-C motif chemokine ligand 8 (CCL8 or MCP-2) biomarker. In certain embodiments, the methods include measuring an expression level of a C-C motif chemokine ligand 13 (CCL13 or MCP-4) biomarker. In certain embodiments, the methods include measuring an expression level of a C-C motif chemokine ligand 15 (CCL15 or MIP-1 delta) biomarker. In certain embodiments, the methods include measuring an expression level of a urokinase plasminogen activator surface receptor (uPAR) biomarker. In certain embodiments, the methods include measuring an expression level of a C-X-C motif chemokine ligand 7 (CXCL7/NAP-2) biomarker. In certain embodiments, the methods include measuring an expression level of I-309, IL-16, IL-1 alpha, MCP-2, MIP-1 delta, and uPAR. In certain embodiments, the methods include measuring an expression level of I-309, IL-16, MCP-4, MIP-1 delta, CXCL7/NAP-2 and uPAR.

In certain embodiments, the methods include identifying the subject as having an aneurysm if the expression level is increased relative to a first reference sample. In certain embodiments, the methods include identifying the subject as having an aneurysm if the expression level is decreased relative to a first reference sample. In certain embodiments, the methods include measuring an expression level of one or more cytokines. In certain embodiments, the methods include determining whether the subject has or is at risk of rupture of the aneurysm. In certain embodiments, the one or more cytokines are selected from the group consisting of RANTES, IL-12 p40/p70, MIP-1α, sTNF.RI, MCP-1, MCP-2, MCP-3, or a combination thereof. In certain embodiments, the methods further include measuring an expression level of RANTES, IL-12 p40/p70, MIP-1α, sTNF.RI, MCP-1, MCP-2, MCP-3, or a combination thereof. In certain embodiments, the one or more cytokines are selected from the group consisting of MCP-2, IL12-p40, sTNF-RI, MIG, IL-1ra, IL1 alpha, or a combination thereof. In certain embodiments, the methods further include measuring an expression level of MCP-2, IL12-p40, sTNF-RI, MIG, IL-1ra, IL1 alpha, or a combination thereof.

In certain embodiments, the cytokine is RANTES. RANTES (also known as CCL5) is an 8 kDa protein classified as a chemotactic cytokine and plays an active role in recruiting leukocytes into inflammatory sites. In certain embodiments, a reduced expression level of RANTES relative to a second reference sample indicates that the subject has or is at risk of rupture of the aneurysm.

As used herein, the term “second reference sample” refers to a control for a biomarker that is to be detected in a biological sample of a subject. In certain embodiments, a second reference sample can be the level of a biomarker detected in an individual that has an unruptured aneurysm. In certain embodiments, a second reference sample can be the level of a biomarker detected in a cohort of individuals that an unruptured aneurysm. In certain embodiments, the second reference sample can be a predetermined level of a biomarker that indicates the presence of an unruptured aneurysm in a subject.

In certain embodiments, the cytokine is IL-12 p40/p70. Interleukin 12 (IL-12) is a pleiotropic cytokine originally identified in the medium of activated human B lymphoblastoid cell lines. IL-12 has two main isoforms (p40 and p70) produced by macrophages and B lymphocytes and has been shown to have multiple effects on T cells and natural killer (NK) cells. In certain embodiments, a reduced expression level of IL-12 p40/p70 relative to a second reference sample indicates that the subject has or is at risk of rupture of the aneurysm.

In certain embodiments, the cytokine is MIP-1α. MIP-1α is a major factor produced by macrophages and monocytes after they are stimulated with proinflammatory cytokines such as IL-1β. It can be expressed by all hematopoietic cells and some tissue cells such as fibroblasts, epithelial cells, vascular smooth muscle cells or platelets upon activation and are crucial for immune responses towards infection and inflammation. The main effect is inflammatory and mainly consists of chemotaxis and trans endothelial migration but cells can be activated to release some bioactive molecules also. In certain embodiments, a reduced expression level of MIP-1α relative to a second reference sample indicates that the subject has or is at risk of rupture of the aneurysm.

In certain embodiments, the cytokine is sTNF.RI. Tumor necrosis factor receptor superfamily member 1A (TNF RI) is a transmembrane protein with an extracellular domain that binds to TNF alpha. This extracellular domain can be proteolytically cleaved to make soluble TNF RI (sTNF.RI). In certain embodiments, a reduced expression level of sTNF.RI relative to a second reference sample indicates that the subject has or is at risk of rupture of the aneurysm.

In certain embodiments, the cytokine is MCP-1. MCP-1 (also known as CCL2) is a small cytokine belonging to the CC chemokine family. MCP-1 is implicated in pathogenesis of several diseases characterized by monocytic infiltrates, such as psoriasis, rheumatoid arthritis and atherosclerosis. MCP-1 is also involved in the neuroinflammatory processes that takes place in the various diseases of the central nervous system (CNS), which are characterized by neuronal degeneration. In certain embodiments, a reduced expression level of MCP-1 relative to a second reference sample indicates that the subject has or is at risk of rupture of the aneurysm.

In certain embodiments, the cytokine is MCP-2. MCP-2 (also known as CCL8) is a small cytokine belonging to the CC chemokine family. MCP-2 is chemotactic for and activates many different immune cells (e.g., T cells and NK cells). MCP-2 is also a potent inhibitor of HIV1 by virtue of its high-affinity binding to the receptor CCR5. In certain embodiments, a reduced expression level of MCP-2 relative to a second reference sample indicates that the subject has or is at risk of rupture of the aneurysm.

In certain embodiments, the cytokine is MCP-3. MCP-3 (also known as CCL7) is a small cytokine belonging to the CC chemokine family. MCP-3 mainly acts as a chemoattractant for several leukocytes, including monocytes, eosinophils, basophils, dendritic cells (DCs), neutrophils, NK cells and activated T lymphocytes. Furthermore, MCP-3 has an influence on diapedesis and extravasation of leukocytes. In certain embodiments, a reduced expression level of MCP-3 relative to a second reference sample indicates that the subject has or is at risk of rupture of the aneurysm.

In certain embodiments, the cytokine is IL-1 alpha. IL-1 alpha (also known as hematopoietin 1) is a cytokine belonging to the interleukin 1 family. The structure of the IL-1 alpha initially synthesized precursor does not contain a signal peptide fragment, however, after processing by the removal of N-terminal amino acids by specific proteases, the resulting peptide is called mature form. IL-1 alpha contributes to the production of inflammation, as well as the promotion of fever and sepsis. IL-1 produced mainly by activated macrophages, as well as neutrophils, epithelial cells, and endothelial cells. It possesses metabolic, physiological, haematopoietic activities, and plays one of the central roles in the regulation of the immune responses. In certain embodiments, a reduced expression level of IL-1 alpha relative to a second reference sample indicates that the subject has or is at risk of rupture of the aneurysm.

In certain embodiments, the cytokine is IL-1RA. IL-1RA is a cytokine belonging to the interleukin 1 family. IL1Ra is secreted by various types of cells including immune cells, epithelial cells, and adipocytes, and is a natural inhibitor of the pro-inflammatory effect of IL-1. IL-1RA inhibits the activities of interleukin 1, alpha (IL1A) and interleukin 1, beta (IL1B), and modulates a variety of interleukin 1 related immune and inflammatory responses. In certain embodiments, a reduced expression level of IL-1RA relative to a second reference sample indicates that the subject has or is at risk of rupture of the aneurysm.

In certain embodiments, the present disclosure provides methods for monitoring a subject's responsiveness to an anti-aneurysm treatment. In certain embodiments, the methods include measuring an expression level of one or more cytokines. In certain embodiments, the methods include determining whether the subject is responsive to the anti-aneurysm treatment. In certain embodiments, the one or more cytokines are selected from the group consisting of RANTES, IL-12 p40/p70, MIP-1α, sTNF.RI, MCP-1, MCP-2, MCP-3, or a combination thereof. In certain embodiments, the methods further include measuring an expression level of RANTES, IL-12 p40/p70, MIP-1α, sTNF.RI, MCP-1, MCP-2, MCP-3, or a combination thereof. In certain embodiments, the one or more cytokines are selected from the group consisting of MCP-2, IL12-p40, sTNF-RI, MIG, IL-1ra, IL1 alpha, or a combination thereof. In certain embodiments, the methods further include measuring an expression level of MCP-2, IL12-p40, sTNF-RI, MIG, IL-1ra, IL1 alpha, or a combination thereof. In certain embodiments, the subject is administered or has been administered with an anti-aneurysm treatment. Non-limiting examples of anti-aneurysms encompassed by the present disclosure are described in Section 3.

In certain embodiments, the cytokine is RANTES. In certain embodiments, a reduced expression level of RANTES relative to a second reference sample indicates that the subject is not responsive to the anti-aneurysm treatment.

In certain embodiments, the cytokine is IL-12 p40/p70. In certain embodiments, a reduced expression level of IL-12 p40/p70 relative to a second reference sample indicates that the subject is not responsive to the anti-aneurysm treatment.

In certain embodiments, the cytokine is MIP-1α. In certain embodiments, a reduced expression level of MIP-1α relative to a second reference sample indicates that the subject is not responsive to the anti-aneurysm treatment.

In certain embodiments, the cytokine is sTNF.RI. In certain embodiments, a reduced expression level of sTNF.RI relative to a second reference sample indicates that the subject is not responsive to the anti-aneurysm treatment.

In certain embodiments, the cytokine is MCP-1. In certain embodiments, an increased expression level of MCP-1 relative to a second reference sample indicates that the subject is not responsive to the anti-aneurysm treatment.

In certain embodiments, the cytokine is MCP-2. In certain embodiments, an increased expression level of MCP-2 relative to a second reference sample indicates that the subject is not responsive to the anti-aneurysm treatment.

In certain embodiments, the cytokine is MCP-3. In certain embodiments, an increased expression level of MCP-3 relative to a second reference sample indicates that the subject is not responsive to the anti-aneurysm treatment.

In certain embodiments, the cytokine is MIP. In certain embodiments, an increased expression level of MIP relative to a second reference sample indicates that the subject is not responsive to the anti-aneurysm treatment.

In certain embodiments, the cytokine is IL-1 alpha. In certain embodiments, an increased expression level of IL-1 alpha relative to a second reference sample indicates that the subject is not responsive to the anti-aneurysm treatment.

In certain embodiments, the cytokine is IL-1RA. In certain embodiments, an increased expression level of IL-1RA relative to a second reference sample indicates that the subject is not responsive to the anti-aneurysm treatment.

In certain embodiments, the presently disclosed methods include measuring the expression level of the biomarkers (e.g., FasL) in a biological sample of the subject. In certain embodiments, the presently disclosed methods include measuring the protein expression level of the biomarkers (e.g., FasL) in a biological sample of the subject.

As used herein, the term “biological sample” or “sample” refers to any sample of biological material obtained from a subject, e.g., a human subject, including a biological fluid, e.g., blood, plasma, serum, urine, sputum, spinal fluid, pleural fluid, nipple aspirates, lymph fluid, fluid of the respiratory, intestinal, and genitourinary tracts, tear fluid, saliva, breast milk, fluid from the lymphatic system, semen, cerebrospinal fluid, intra-organ system fluid, ascitic fluid, cyst fluid, amniotic fluid, bronchoalveolar fluid, biliary fluid, or any combinations thereof. In certain embodiments, the biological sample is a blood sample. In certain embodiments, the biological sample is a plasma sample. In certain embodiments, the biological sample is a serum sample. In certain embodiments, the biological sample is a cerebrospinal fluid sample.

Any suitable methods known in the art for measuring protein levels can be used with the presently disclosed methods. These methods include, but are not limited to, mass spectrometry techniques, 1-D or 2-D gel-based analysis systems, chromatography, enzyme linked immunosorbent assays (ELISAs), flow cytometry, radioimmunoassays (RIA), enzyme immunoassays (EIA), Western Blotting, immunoprecipitation, and immunohistochemistry. These methods use antibodies, or antibody equivalents, to detect protein. Antibody arrays or protein chips can also be employed.

ELISA and RIA procedures can be conducted such that a protein standard is labeled (with a radioisotope such as ¹²⁵I or ³⁵S, or an assayable enzyme, such as horseradish peroxidase or alkaline phosphatase), and, together with the unlabeled sample, brought into contact with the corresponding antibody, whereon a second antibody is used to bind the first, and radioactivity or the immobilized enzyme assayed (competitive assay). Alternatively, the protein can react with the corresponding immobilized antibody, radioisotope, or enzyme-labeled anti-marker antibody is allowed to react with the system, and radioactivity or the enzyme assayed (ELISA-sandwich assay). Other conventional methods can also be employed as suitable.

The above techniques can be conducted essentially as a “one-step” or “two-step” assay. A “one-step” assay involves contacting antigen with immobilized antibody and, without washing, contacting the mixture with labeled antibody. A “two-step” assay involves washing before contacting, the mixture with labeled antibody. Other conventional methods can also be employed as suitable.

In certain embodiments, the detection of a biomarker from a biological sample includes contacting the sample with an antibody or variant (e.g., fragment) thereof which selectively binds the biomarker, and detecting whether the antibody or variant thereof is bound to the sample. The method can further include contacting the sample with a second antibody, e.g., a labeled antibody. The method can further include one or more washing, e.g., to remove one or more reagents.

It can be desirable to immobilize one component of the assay system on a support, thereby allowing other components of the system to be brought into contact with the component and readily removed without laborious and time-consuming labor. It is possible for a second phase to be immobilized away from the first, but one phase is usually sufficient.

It is possible to immobilize the enzyme itself on a support, but if solid-phase enzyme is required, then this is generally best achieved by binding to antibody and affixing the antibody to a support, models, and systems for which are well-known in the art. Simple polyethylene can provide a suitable support.

Enzymes employable for labeling are not particularly limited but can be selected from the members of the oxidase group, for example. These catalyze the production of hydrogen peroxide by reaction with their substrates, and glucose oxidase is often used for its good stability, ease of availability and cheapness, as well as the ready availability of its substrate (glucose). Activity of the oxidase can be assayed by measuring the concentration of hydrogen peroxide formed after reaction of the enzyme-labeled antibody with the substrate under controlled conditions well-known in the art.

Other techniques can be used to detect a protein marker according to a practitioner's preference based upon the present disclosure. One such technique is Western blotting (Towbin et al., Proc. Nat. Acad. Sci. 76:4350 (1979)), wherein a suitably treated sample is run on an SDS-PAGE gel before being transferred to a solid support, such as a nitrocellulose filter. Antibodies (unlabeled) are then brought into contact with the support and assayed by a secondary immunological reagent, such as labeled protein A or anti-immunoglobulin (suitable labels including ¹²⁵I, horseradish peroxidase, and alkaline phosphatase). Chromatographic detection can also be used.

Other machine or auto imaging systems can also be used to measure immunostaining results for the marker. As used herein, “quantitative” immunohistochemistry refers to an automated method of scanning and scoring samples that have undergone immunohistochemistry, to identify and quantitate the presence of a specified marker, such as an antigen or other protein. The score given to the sample is a numerical representation of the intensity of the immunohistochemical staining of the sample and represents the amount of target marker present in the sample. As used herein, Optical Density (OD) is a numerical score that represents intensity of staining. As used herein, semi-quantitative immunohistochemistry refers to scoring of immunohistochemical results by human eye, where a trained operator ranks results numerically (e.g., as 1, 2 or 3).

Various automated sample processing, scanning, and analysis systems suitable for use with immunohistochemistry are available in the art. Such systems can include automated staining (see, e.g., the Benchmark system, Ventana Medical Systems, Inc.) and microscopic scanning, computerized image analysis, serial section comparison (to control for variation in the orientation and size of a sample), digital report generation, and archiving and tracking of samples (such as slides on which tissue sections are placed). Cellular imaging systems are commercially available that combine conventional light microscopes with digital image processing systems to perform quantitative analysis on cells and tissues, including immunostained samples.

Antibodies against biomarkers can also be used for imaging purposes, for example, to detect the presence of any of the biomarkers disclosed herein in a sample obtained from a recipient's blood. Suitable labels include radioisotopes, iodine (¹²⁵I, ¹²¹I), carbon (¹⁴C), sulfur (³⁵S), tritium (³H), indium (¹¹²In), and technetium (⁹⁹mTc), fluorescent labels, such as fluorescein, rhodamine, and biotin. Immunoenzymatic interactions can be visualized using different enzymes such as peroxidase, alkaline phosphatase, or different chromogens such as DAB, AEC, or Fast Red.

Antibodies for use in the present disclosure include any antibody, whether natural or synthetic, full length or a fragment thereof, monoclonal, or polyclonal, that binds sufficiently strongly and specifically to the marker to be detected. An antibody can have a K_{d} of at most about 10⁻⁶M, 10⁻⁷M, 10⁻⁸M, 10⁻⁹M, 10⁻¹⁰M, 10⁻¹¹M, 10⁻¹²M. The phrase “specifically binds” refers to binding of, for example, an antibody to an epitope or antigen or antigenic determinant in such a manner that binding can be displaced or competed with a second preparation of identical or similar epitope, antigen, or antigenic determinant.

Antibodies and derivatives thereof that can be used encompasses polyclonal or monoclonal antibodies, chimeric, human, humanized, primatized (CDR-grafted), veneered or single-chain antibodies, phase produced antibodies (e.g., from phage display libraries), as well as functional binding fragments, of antibodies. For example, antibody fragments capable of binding to a marker, or portions thereof, including, but not limited to Fv, Fab, Fab′ and F(ab′)2 fragments can be used. Such fragments can be produced by enzymatic cleavage or by recombinant techniques. For example, papain or pepsin cleavage can generate Fab or F(ab′)2 fragments, respectively. Other proteases with the requisite substrate specificity can also be used to generate Fab or F(ab′)2 fragments. Antibodies can also be produced in a variety of truncated forms using antibody genes in which one or more stop codons have been introduced upstream of the natural stop site. For example, a chimeric gene encoding a F(ab′)2 heavy chain portion can be designed to include DNA sequences encoding the CH, domain and hinge region of the heavy chain. In certain embodiments, the antibodies can be conjugated to quantum dots.

In addition, a biomarker can be detected using Mass Spectrometry such as MALDI/TOF (time-of-flight), SELDI/TOF, liquid chromatography-mass spectrometry (LC-MS), gas chromatography-mass spectrometry (GC-MS), high performance liquid chromatography-mass spectrometry (HPLC-MS), capillary electrophoresis-mass spectrometry, nuclear magnetic resonance spectrometry, or tandem mass spectrometry (e.g., MS/MS, MS/MS/MS, ESI-MS/MS, etc.). See, for example, U.S. Patent Application Nos: 2003/0199001, 2003/0134304, 2003/0077616, which are herein incorporated by reference.

Mass spectrometry methods are well known in the art and have been used to detect biomolecules, such as proteins (see, e.g., Li et al. (2000) Tibtech 18:151-160; Rowley et al. (2000) Methods 20: 383-397; and Kuster and Mann (1998) Curr. Opin. Structural Biol. 8: 393-400). Further, mass spectrometric techniques have been developed that permit at least partial de novo sequencing of isolated proteins. Chait et al., Science 262:89-92 (1993); Keough et al., Proc. Natl. Acad. Sci. USA. 96:7131-6 (1999); reviewed in Bergman, EXS 88:133-44 (2000).

In certain embodiments, a gas phase ion spectrophotometer can be used. In other embodiments, laser-desorption/ionization mass spectrometry is used to analyze the sample. Modem laser desorption/ionization mass spectrometry (“LDI-MS”) can be practiced in two main variations: matrix assisted laser desorption/ionization (“MALDI”) mass spectrometry and surface-enhanced laser desorption/ionization (“SELDI”). In MALDI, the analyte is mixed with a solution containing a matrix, and a drop of the liquid is placed on the surface of a substrate. The matrix solution then co-crystallizes with the biological molecules. The substrate is inserted into the mass spectrometer. Laser energy is directed to the substrate surface where it desorbs and ionizes the biological molecules without significantly fragmenting them. However, MALDI has limitations as an analytical tool. It does not provide means for fractionating the sample, and the matrix material can interfere with detection, especially for low molecular weight analytes. See, e.g., U.S. Pat. No. 5,118,937 (Hillenkamp et al.), and U.S. Pat. No. 5,045,694 (Beavis & Chait).

For additional information regarding mass spectrometers, see, e.g., Principles of Instrumental Analysis, 3rd edition. Skoog, Saunders College Publishing, Philadelphia, 1985; and Kirk-Othmer Encyclopedia of Chemical Technology, 4th ed. Vol. 15 (John Wiley & Sons, New York 1995), pp. 1071-1094.

Detection of the presence of a marker or other substances can involve detection of signal intensity. This, in turn, can reflect the quantity and character of a polypeptide bound to the substrate. For example, in certain embodiments, the signal strength of peak values from spectra of a first sample and a second sample can be compared (e.g., visually, by computer analysis etc.), to determine the relative amounts of a particular marker. Software programs such as the Biomarker Wizard program (Ciphergen Biosystems, Inc., Fremont, Calif.) can be used to aid in analyzing mass spectra. The mass spectrometers and their techniques are well known to those of skill in the art.

Any person skilled in the art understands, the components of a mass spectrometer (e.g., desorption source, mass analyzer, detect, etc.) and varied sample preparations can be combined with other suitable components or preparations described herein, or to those known in the art. For example, in certain embodiments, a control sample can contain heavy atoms (e.g., ¹³C) thereby permitting the test sample to be mixed with the known control sample in the same mass spectrometry run.

In certain embodiments, a laser desorption time-of-flight (TOF) mass spectrometer is used. In laser desorption mass spectrometry, a substrate with a bound marker is introduced into an inlet system. The marker is desorbed and ionized into the gas phase by laser from the ionization source. The ions generated are collected by an ion optic assembly, and then in a time-of-flight mass analyzer, ions are accelerated through a short high voltage field and let drift into a high vacuum chamber. At the far end of the high vacuum chamber, the accelerated ions strike a sensitive detector surface at a different time. Since the time-of-flight is a function of the mass of the ions, the elapsed time between ion formation and ion detector impact can be used to identify the presence or absence of molecules of specific mass to charge ratio.

### 3. Web-Based and/or Mobile-Based Software

In certain embodiments, the present disclosure provides a web-based and/or mobile-based software to properly assign risk stratification and need for surgery for patients with known cerebral aneurysms. The software can be implemented by one or more statistical packages (e.g., R statistical package). The software leverages one or more of patient demographics, co-morbidities, aneurysm size, aneurysm location, or inflammatory cytokine data to properly assign risk of rupture and need for surgery to patients on individual basis.

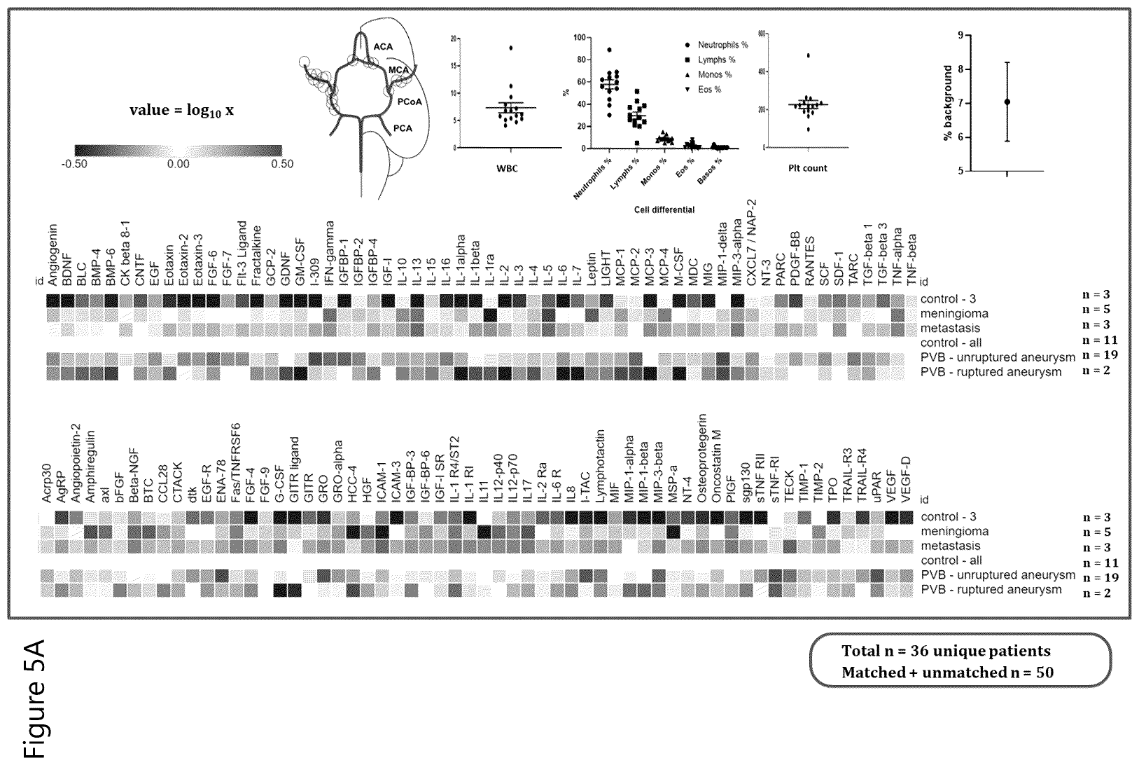

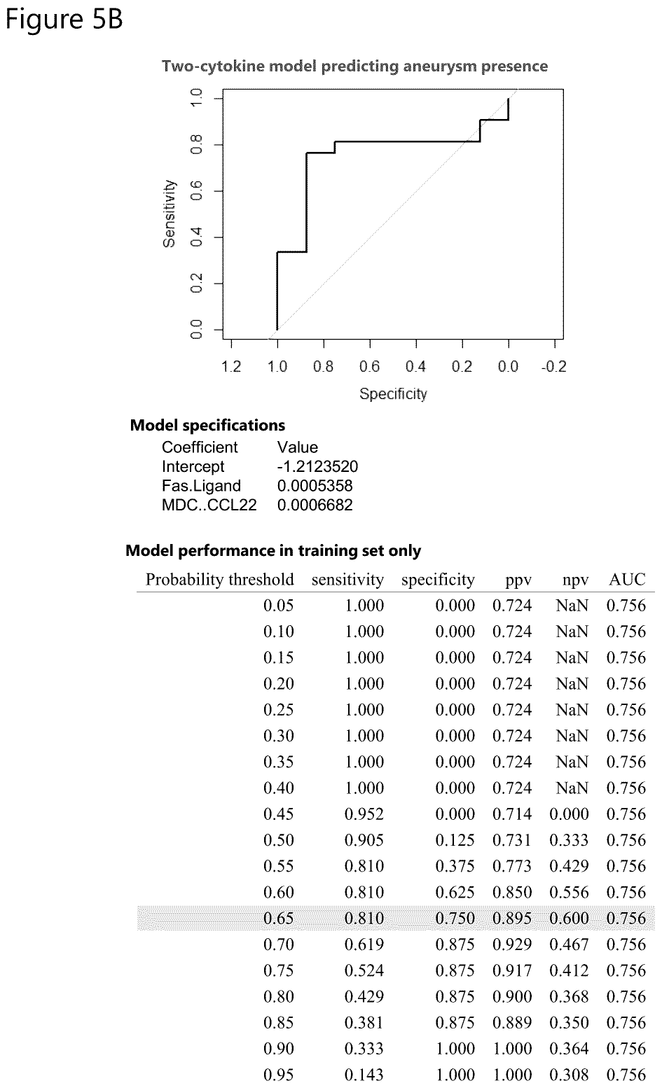

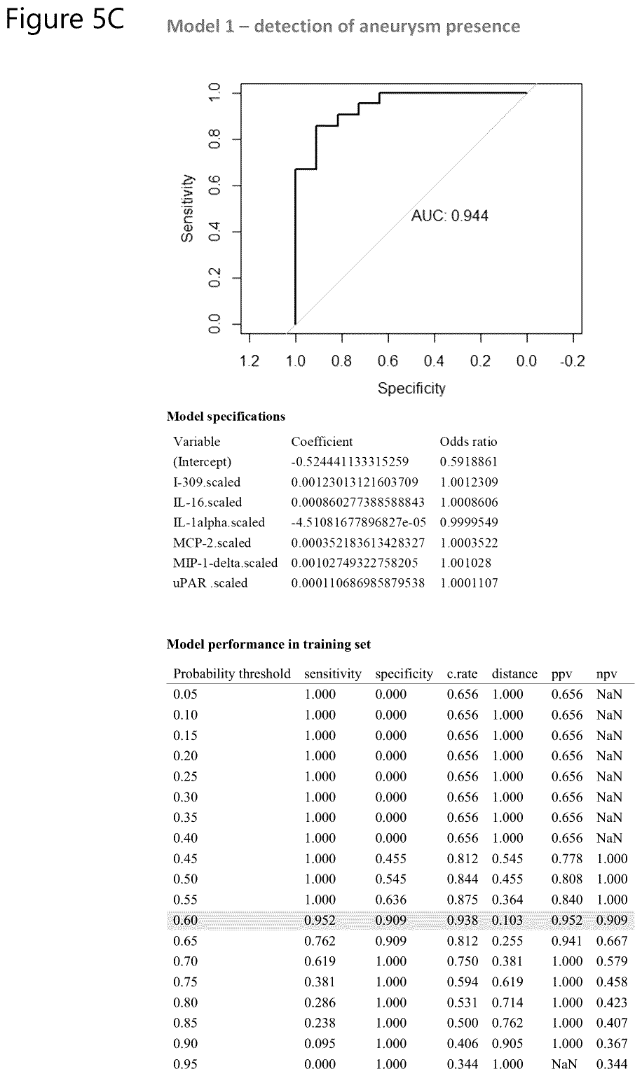

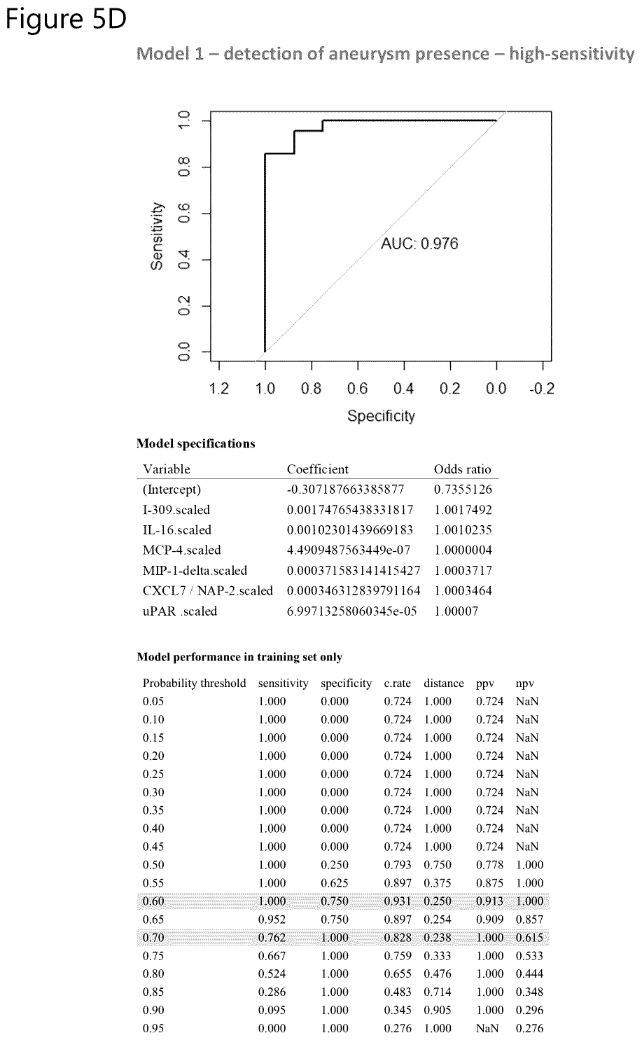

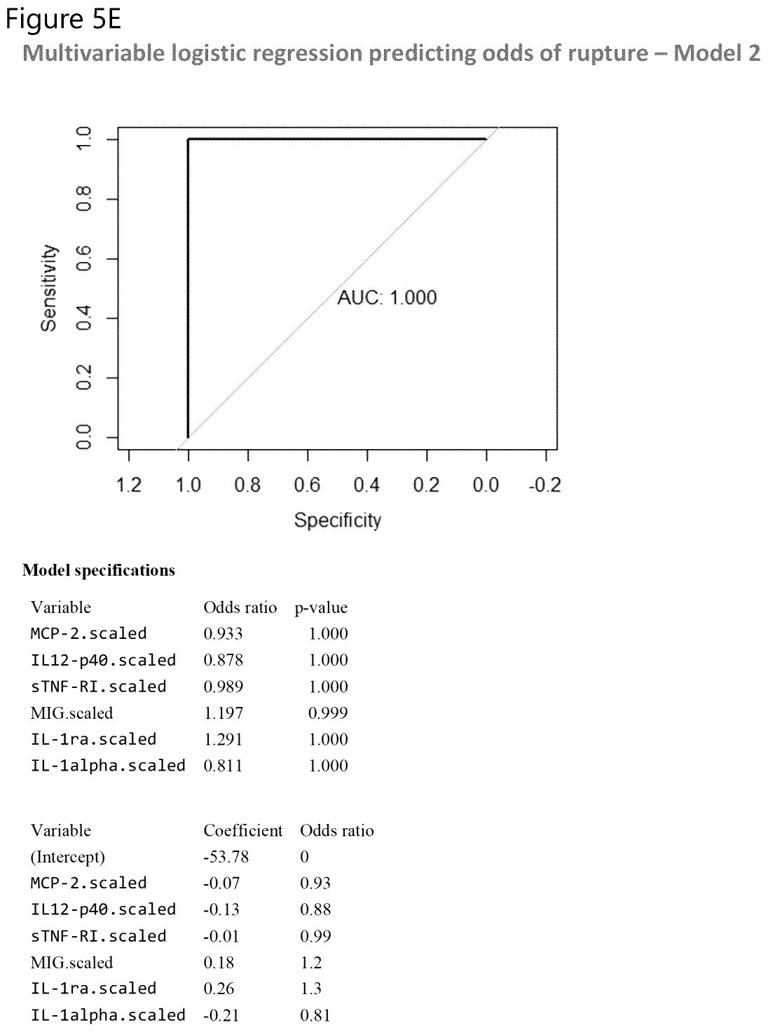

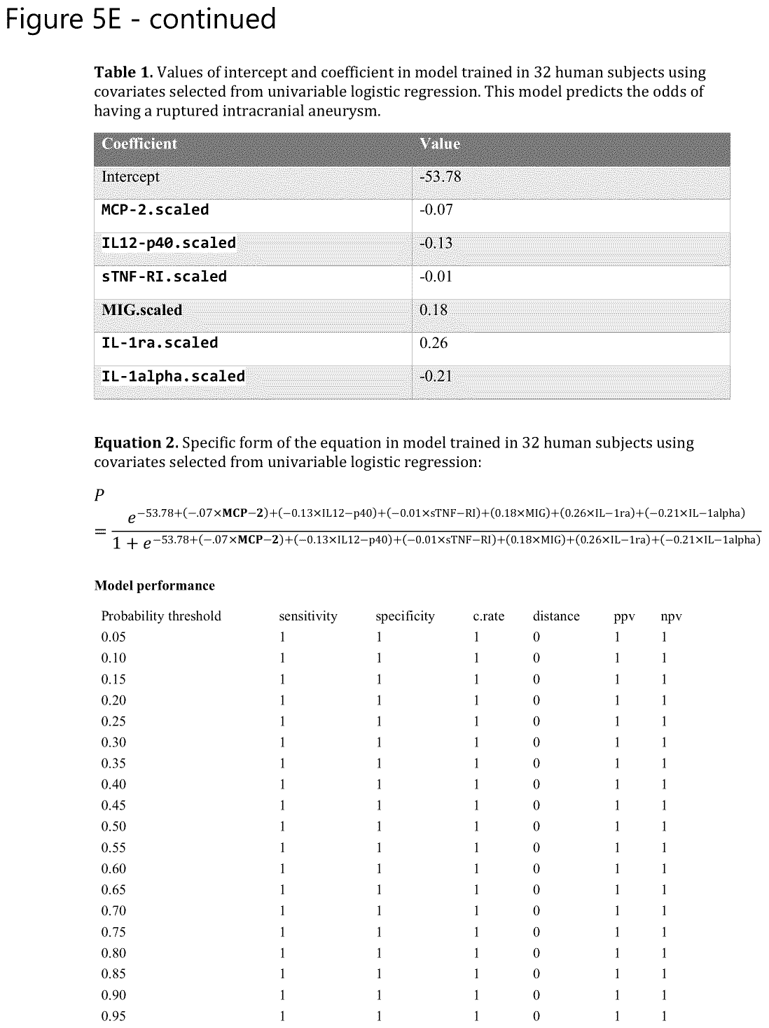

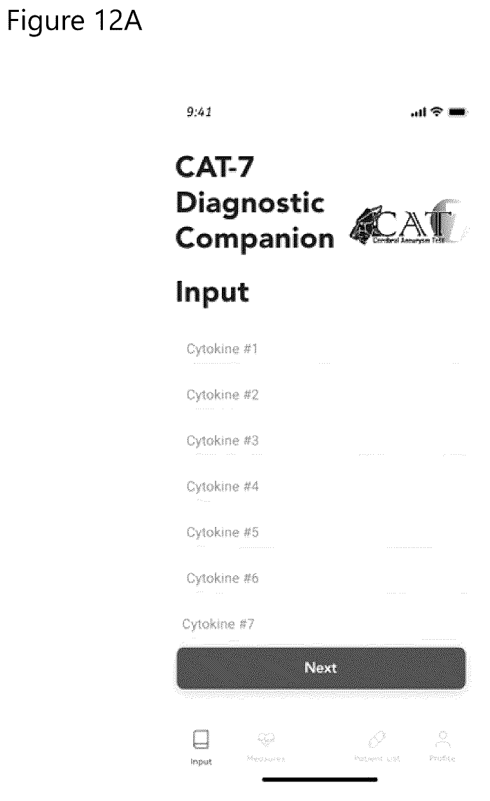

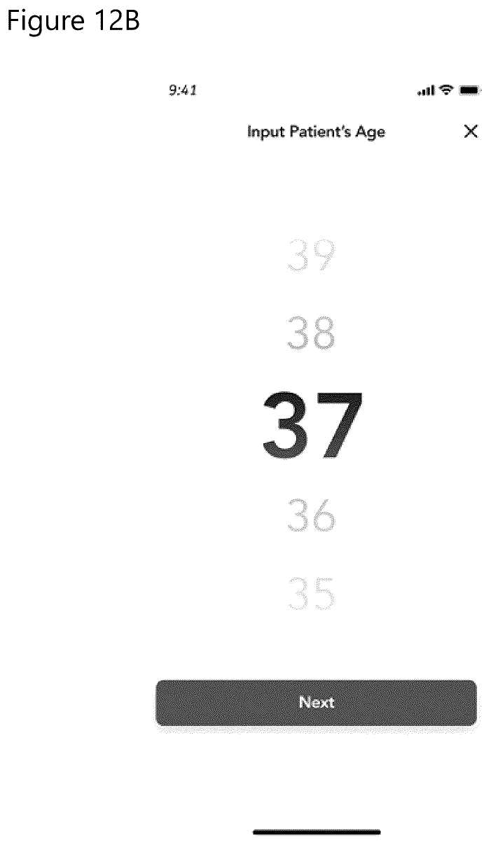

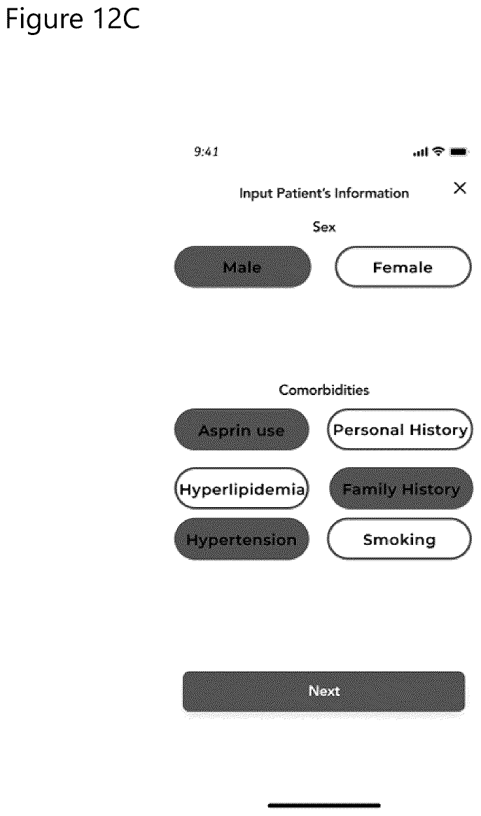

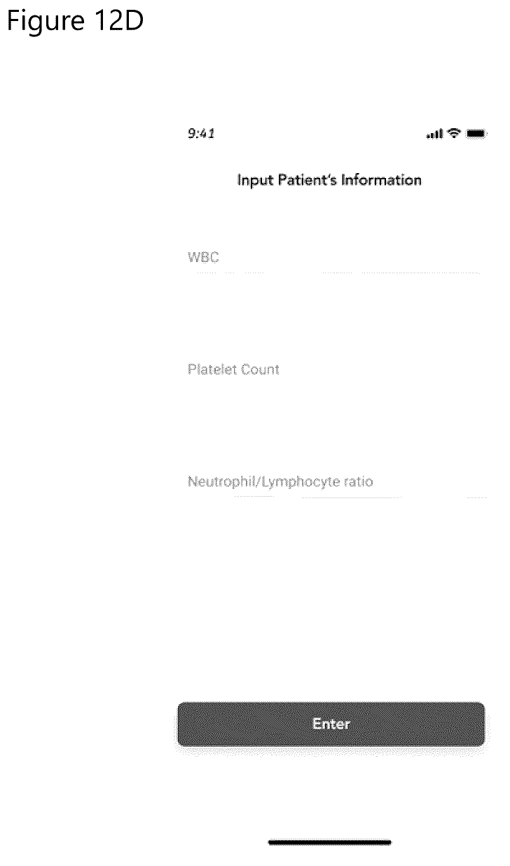

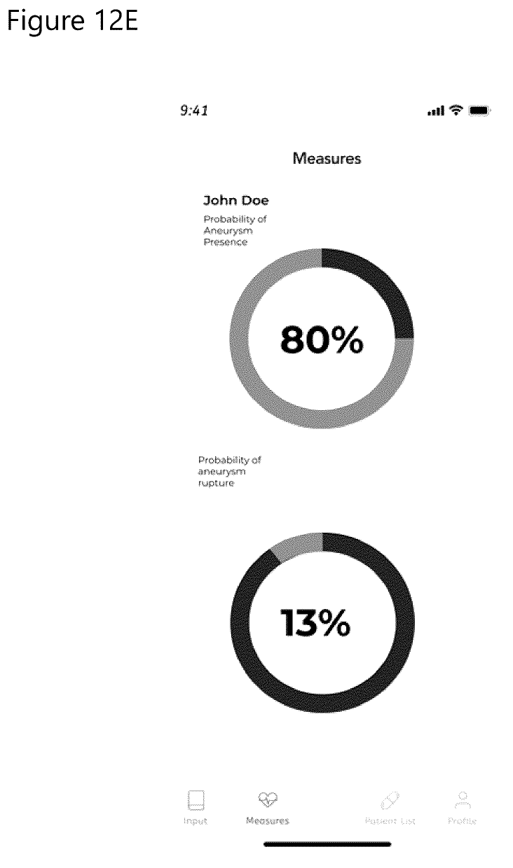

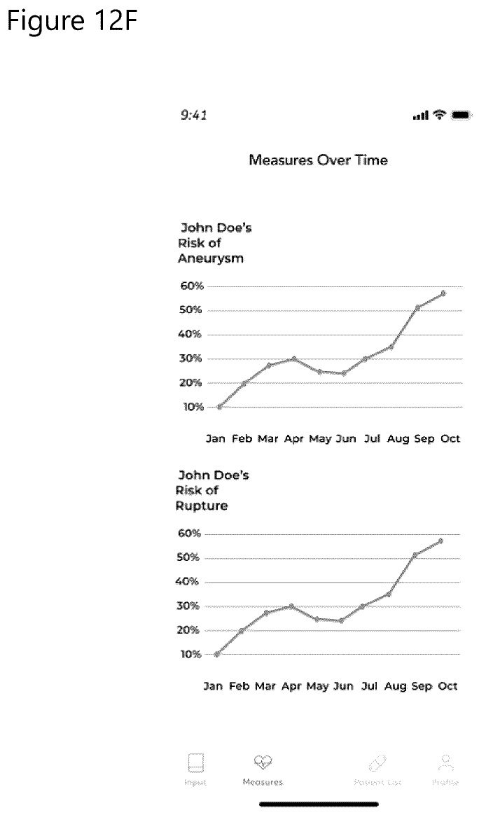

FIG. 12A shows a user interface of the software for inputting cytokines associated with the patient. A user, e.g., a practitioner, can select the “input” function displayed at the bottom of the user interface to start the inputting process. As an example and not by way of limitation, the inputting process may start with inputting one or more cytokines, as shown in FIG. 12A. The user can input multiple cytokines including I-309, IL-16, IL-1 alpha, MCP-2, MIP-1 delta, and uPAR, or a combination thereof, as shown in FIG. 5C, or more. The user can select the “measure” function displayed at the bottom of the user interface to get various measures associated with aneurysm presence and aneurysm rupture of a patient. The user can select the “patient list” function displayed at the bottom of the user interface to see the patients whom the user has a record of within the software. The user can additionally select the “profile” function displayed at the bottom of the user interface to see the user's profile registered with the software (e.g., settings). After inputting the cytokines, the user can select “next” for inputting additional information of the patient.

FIG. 12B shows a user interface of the software for inputting the patient's age. The user can scroll up and down to select the patient's age. For example, 37 is selected as the patient's age in FIG. 12B. After inputting the patient's age, the user can select “next” for inputting additional information of the patient.

FIG. 12C shows a user interface of the software for inputting the patient's information. The user can input the patient's additional information such as sex and comorbidities, as shown in FIG. 12C. For example, the user selected the patient's sex as male. As another example, the user selected the patient's comorbidities including use of medications (e.g., aspirin), family history, and hypertension. After inputting such additional information, the user can select “next” for inputting more information.

FIG. 12D shows a user interface of the software for inputting the patient's additional information. For example, the user can input the patient's white blood cells count (WBC), platelet count, and neutrophil/lymphocyte ratio, as shown in FIG. 12D. After inputting all the required information, the user can enter them by selecting “enter”, as shown in FIG. 12D.

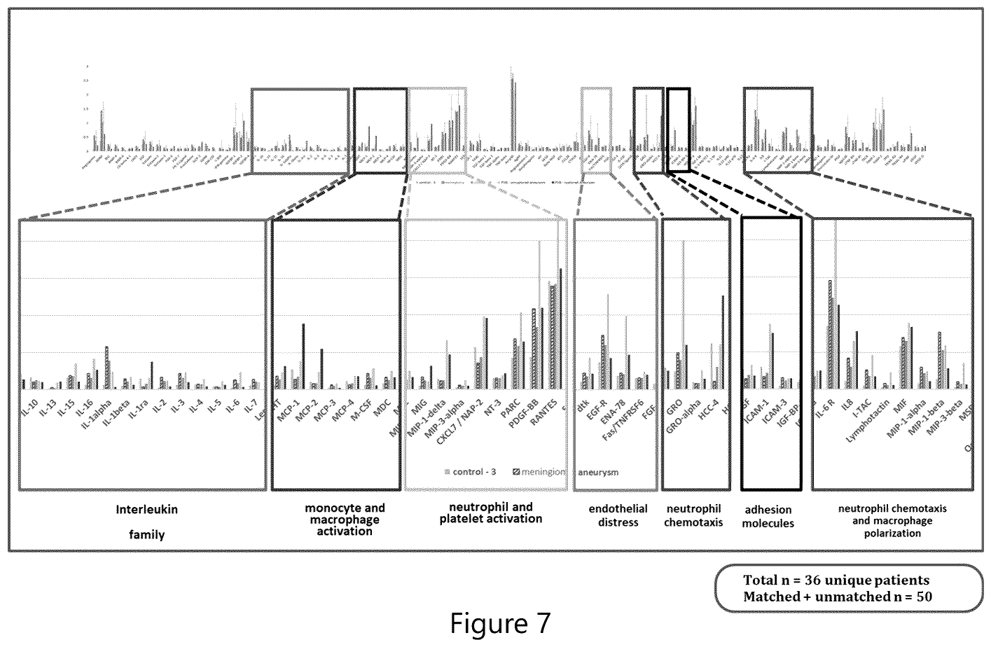

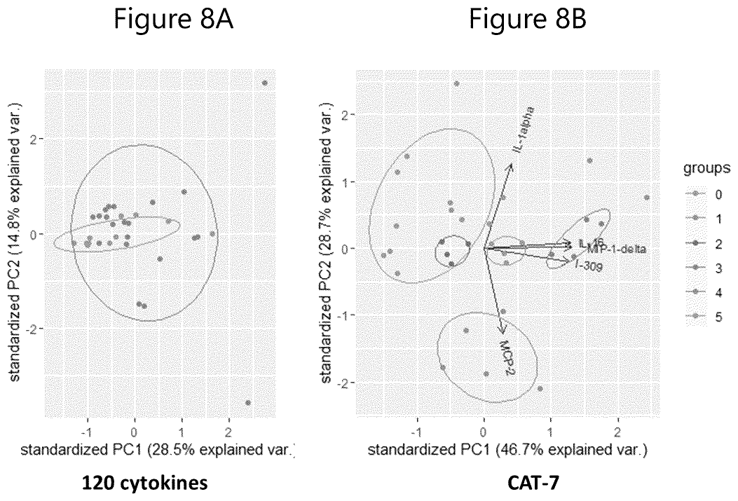

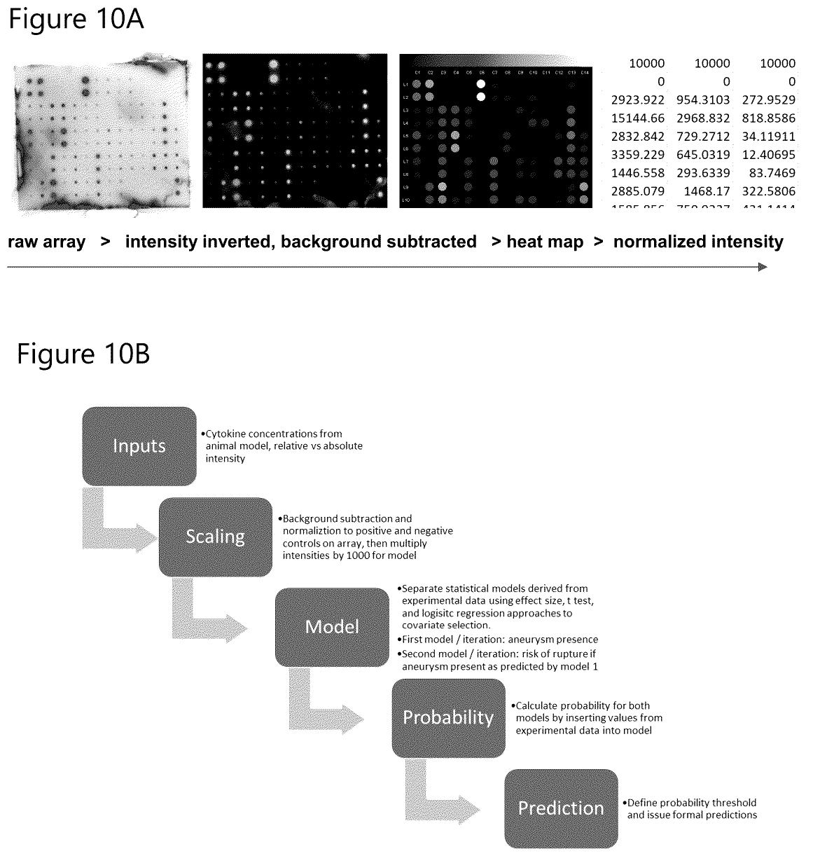

Once the required information is entered into the software, the software can determine various measures for the patients regarding aneurysm presence and aneurysm rupture. In certain embodiments, the software uses a diagnostic test to determine the probability of a patient harboring an aneurysm as well as their risk of potential catastrophic rupture. Building a diagnostic test comprises data extraction, model building, and probability calculation. The diagnostic test was developed using an interactive approach with retrospective human cytokine data (FIG. 7) and sample stratification based on t-SNE inflammatory cytokine analysis (FIGS. 8A and 8B) and applied it as shown in FIGS. 10A and 10B (peripheral blood n=21, aneurysm dome n=3, reference serum n=3, total controls=11).

In certain embodiments, model building and probability calculation can be based on the following equation 1.

### Equation 1. General Form of Probability Equation Based on a LASSO Method:

$$P = \frac{{e}^{{β}_{0} + {β}_{1} {X}_{1} ⋯ + {β}_{n} {X}_{n}}}{1 + {e}^{{β}_{0} + {β}_{1} {X}_{1} ⋯ + {β}_{n} {X}_{n}}}$$

where P is the probability of the event, Po is the value of the intercept from the model, β₁ is the coefficient of the first covariate from the model, and X₁ is the value of the first covariate. This equation extends for as many covariates (e.g., up to Xₙ) as are included in the equation. The first model developed, based on data collected from subjects at a first time period and the equation 1, predicts the likelihood of aneurysm presence. As an example and not by way of limitation, the first model can be based on the following equation 2.
   Equation 2. Specific Form of the Equation in Model Data Using Covariates Selected from a LASSO Method:

$$P = \frac{{e}^{{β}_{0} + {β}_{1} \times I 309 + {β}_{2} \times IL 16 + {β}_{3} \times IL 1 + {β}_{4} \times MCP 2 + {β}_{5} \times MIP 1. delta + {β}_{6} \times uPAR}}{1 + {e}^{{β}_{0} + {β}_{1} \times I 309 + {β}_{2} \times IL 16 + {β}_{3} \times IL 1 + {β}_{4} \times MCP 2 + {β}_{5} \times MIP 1. delta + {β}_{6} \times uPAR}}$$

where β₀ and β1 are determined based on the collected data and I309, IL-16, IL-1, MCP-2, MIP-1 delta, and uPAR are specific to the patient.

By calculating the equation, the first model determines the probability of the patient harboring an aneurysm. The second model developed, based on data collected from subjects at a second time period and the Equation 1, predicts likelihood of ruptured aneurysm or impending rupture. As an example and not by way of limitation, the second model can be based on the following equation 3.

Equation 3. Specific Form of the Equation in Model Using Covariates Selected from Univariable or Multivariable Logistic Regression:

$$P = \frac{{e}^{{β}_{0} + ( {β}_{1} \times MCP 2 ) + ( {β}_{2} \times IL 12 , p 40 ) + ( {β}_{3} \times sTNF . RI ) + ( {β}_{4} \times MIG ) + ( {β}_{5} \times IL 1. RA ) + ( {β}_{6} \times IL 1. alpha )}}{1 + {e}^{{β}_{0} + ( {β}_{1} \times MCP 2 ) + ( {β}_{2} \times IL 12 , p 40 ) + ( {β}_{3} \times sTNF . RI ) + ( {β}_{4} \times MIG ) + ( {β}_{5} \times IL 1. RA ) + ( {β}_{6} \times IL 1. alpha )}}$$

where β₀ to β₇ are determined based on the collected data and MCP-2, IL.12.p40, sTNF.RI, MIG, IL-1 RA, and/or IL-1 alpha are provided by the user (e.g., via the software).

By calculating the equation, the second model determines the probability of the patient harboring a ruptured aneurysm or an aneurysm with impending rupture.

Although the disclosure describes using particular models to determine probabilities of aneurysm presence and aneurysm rupture in particular manners, this disclosure contemplates using any suitable model to determine probabilities of aneurysm presence and aneurysm rupture in any suitable manner. As an example and not by way of limitation, the model may be a machine-learning model based on one or more of a convolutional neural network, a support vector machine (SVM), or a regression model. The machine-learning model may be trained based on training data collected from a plurality of subjects (e.g., patients). The training data may comprise information (e.g., cytokines) associated with the subjects and corresponding indications of aneurysm presence and/or aneurysm rupture. As a result, when inputting a new subject's information to the machine-learning model, the machine-learning model is able to predict the probabilities of aneurysm presence and aneurysm rupture for the new subject.

FIG. 12E shows a user interface displaying the predicted probability of aneurysm presence and the probability of aneurysm rupture of the patient. After the diagnostic test integrated in the software determine the probabilities of aneurysm presence and aneurysm rupture, the software outputs them via the user interface in the section of “measures”, as shown in FIG. 12E. For example, the patient's name may be John Doe. The probability of aneurysm presence in the patient is 80%, as determined by the diagnostic test. The probability of aneurysm rupture in the patient is 13%, as determined by the diagnostic test.

FIG. 12F shows a user interface displaying the measures of risk of aneurysm and risk of rupture over time. The software can further output the measures for a patient over time. The measures over time are tracked measures from hypothetical “repeated” testing of risk of aneurysm and risk of rupture. For example, as shown in FIG. 12F, John Doe's risk of aneurysm presence progresses from 10% to almost 60% from January to October. As another example, as shown in FIG. 12F, John Doe's risk of aneurysm rupture progresses from 10% to almost 60% from January to October.

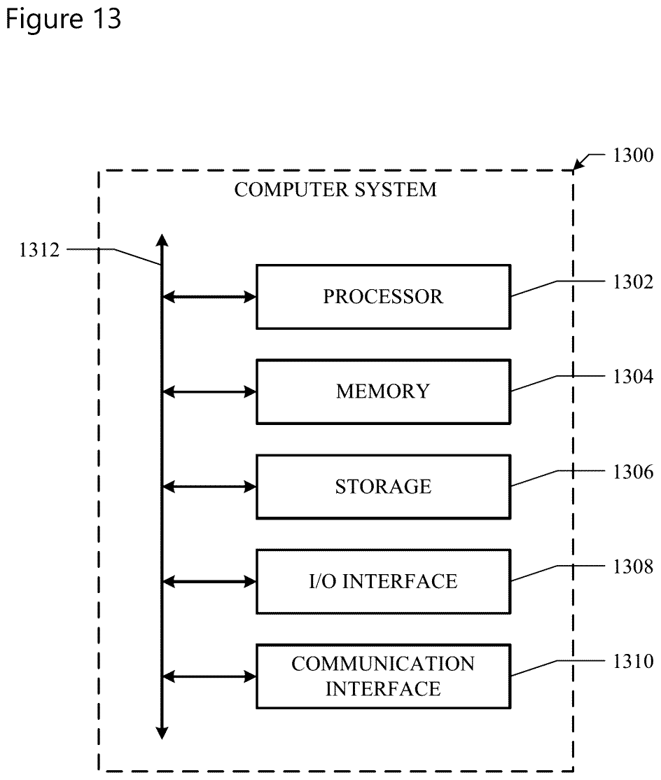

FIG. 13 shows an example computer system 1300. In particular embodiments, one or more computer systems 1300 perform one or more steps of one or more methods described or illustrated herein. In particular embodiments, one or more computer systems 1300 provide functionality described or illustrated herein. In particular embodiments, software running on one or more computer systems 1300 performs one or more steps of one or more methods described or illustrated herein or provides functionality described or illustrated herein. Particular embodiments include one or more portions of one or more computer systems 1300. Herein, reference to a computer system may encompass a computing device, and vice versa, where appropriate. Moreover, reference to a computer system may encompass one or more computer systems, where appropriate.

This disclosure contemplates any suitable number of computer systems 1300. This disclosure contemplates computer system 1300 taking any suitable physical form. As example and not by way of limitation, computer system 1300 may be an embedded computer system, a system-on-chip (SOC), a single-board computer system (SBC) (such as, for example, a computer-on-module (COM) or system-on-module (SOM)), a desktop computer system, a laptop or notebook computer system, an interactive kiosk, a mainframe, a mesh of computer systems, a mobile telephone, a personal digital assistant (PDA), a server, a tablet computer system, or a combination of two or more of these. Where appropriate, computer system 1300 may include one or more computer systems 1300; be unitary or distributed; span multiple locations; span multiple machines; span multiple data centers; or reside in a cloud, which may include one or more cloud components in one or more networks. Where appropriate, one or more computer systems 1300 may perform without substantial spatial or temporal limitation one or more steps of one or more methods described or illustrated herein. As an example and not by way of limitation, one or more computer systems 1300 may perform in real time or in batch mode one or more steps of one or more methods described or illustrated herein. One or more computer systems 1300 may perform at different times or at different locations one or more steps of one or more methods described or illustrated herein, where appropriate.

In particular embodiments, computer system 1300 includes a processor 1302, memory 1304, storage 1306, an input/output (I/O) interface 1308, a communication interface 1310, and a bus 1312. Although this disclosure describes and illustrates a particular computer system having a particular number of particular components in a particular arrangement, this disclosure contemplates any suitable computer system having any suitable number of any suitable components in any suitable arrangement.

In particular embodiments, processor 1302 includes hardware for executing instructions, such as those making up a computer program. As an example and not by way of limitation, to execute instructions, processor 1302 may retrieve (or fetch) the instructions from an internal register, an internal cache, memory 1304, or storage 1306; decode and execute them; and then write one or more results to an internal register, an internal cache, memory 1304, or storage 1306. In particular embodiments, processor 1302 may include one or more internal caches for data, instructions, or addresses. This disclosure contemplates processor 1302 including any suitable number of any suitable internal caches, where appropriate. As an example and not by way of limitation, processor 1302 may include one or more instruction caches, one or more data caches, and one or more translation lookaside buffers (TLBs). Instructions in the instruction caches may be copies of instructions in memory 1304 or storage 1306, and the instruction caches may speed up retrieval of those instructions by processor 1302. Data in the data caches may be copies of data in memory 1304 or storage 1306 for instructions executing at processor 1302 to operate on; the results of previous instructions executed at processor 1302 for access by subsequent instructions executing at processor 1302 or for writing to memory 1304 or storage 1306; or other suitable data. The data caches may speed up read or write operations by processor 1302. The TLBs may speed up virtual-address translation for processor 1302. In particular embodiments, processor 1302 may include one or more internal registers for data, instructions, or addresses. This disclosure contemplates processor 1302 including any suitable number of any suitable internal registers, where appropriate. Where appropriate, processor 1302 may include one or more arithmetic logic units (ALUs); be a multi-core processor; or include one or more processors 1302. Although this disclosure describes and illustrates a particular processor, this disclosure contemplates any suitable processor.

In particular embodiments, memory 1304 includes main memory for storing instructions for processor 1302 to execute or data for processor 1302 to operate on. As an example and not by way of limitation, computer system 1300 may load instructions from storage 1306 or another source (such as, for example, another computer system 1300) to memory 1304. Processor 1302 may then load the instructions from memory 1304 to an internal register or internal cache. To execute the instructions, processor 1302 may retrieve the instructions from the internal register or internal cache and decode them. During or after execution of the instructions, processor 1302 may write one or more results (which may be intermediate or final results) to the internal register or internal cache.

Processor 1302 may then write one or more of those results to memory 1304. In particular embodiments, processor 1302 executes only instructions in one or more internal registers or internal caches or in memory 1304 (as opposed to storage 1306 or elsewhere) and operates only on data in one or more internal registers or internal caches or in memory 1304 (as opposed to storage 1306 or elsewhere). One or more memory buses (which may each include an address bus and a data bus) may couple processor 1302 to memory 1304. Bus 1312 may include one or more memory buses, as described below. In particular embodiments, one or more memory management units (MMUs) reside between processor 1302 and memory 1304 and facilitate accesses to memory 1304 requested by processor 1302. In particular embodiments, memory 1304 includes random access memory (RAM). This RAM may be volatile memory, where appropriate. Where appropriate, this RAM may be dynamic RAM (DRAM) or static RAM (SRAM). Moreover, where appropriate, this RAM may be single-ported or multi-ported RAM. This disclosure contemplates any suitable RAM. Memory 1304 may include one or more memories 1304, where appropriate. Although this disclosure describes and illustrates particular memory, this disclosure contemplates any suitable memory.

In particular embodiments, storage 1306 includes mass storage for data or instructions. As an example and not by way of limitation, storage 1306 may include a hard disk drive (HDD), a floppy disk drive, flash memory, an optical disc, a magneto-optical disc, magnetic tape, or a Universal Serial Bus (USB) drive or a combination of two or more of these. Storage 1306 may include removable or non-removable (or fixed) media, where appropriate. Storage 1306 may be internal or external to computer system 1300, where appropriate. In particular embodiments, storage 1306 is non-volatile, solid-state memory. In particular embodiments, storage 1306 includes read-only memory (ROM). Where appropriate, this ROM may be mask-programmed ROM, programmable ROM (PROM), erasable PROM (EPROM), electrically erasable PROM (EEPROM), electrically alterable ROM (EAROM), or flash memory or a combination of two or more of these. This disclosure contemplates mass storage 1306 taking any suitable physical form. Storage 1306 may include one or more storage control units facilitating communication between processor 1302 and storage 1306, where appropriate. Where appropriate, storage 1306 may include one or more storages 1306. Although this disclosure describes and illustrates particular storage, this disclosure contemplates any suitable storage.

In particular embodiments, I/O interface 1308 includes hardware, software, or both, providing one or more interfaces for communication between computer system 1300 and one or more I/O devices. Computer system 1300 may include one or more of these I/O devices, where appropriate. One or more of these I/O devices may enable communication between a person and computer system 1300. As an example and not by way of limitation, an I/O device may include a keyboard, keypad, microphone, monitor, mouse, printer, scanner, speaker, still camera, stylus, tablet, touch screen, trackball, video camera, another suitable I/O device or a combination of two or more of these. An I/O device may include one or more sensors. This disclosure contemplates any suitable I/O devices and any suitable I/O interfaces 1308 for them. Where appropriate, I/O interface 1308 may include one or more device or software drivers enabling processor 1302 to drive one or more of these I/O devices. I/O interface 1308 may include one or more I/O interfaces 1308, where appropriate. Although this disclosure describes and illustrates a particular I/O interface, this disclosure contemplates any suitable I/O interface.

In particular embodiments, communication interface 1310 includes hardware, software, or both providing one or more interfaces for communication (such as, for example, packet-based communication) between computer system 1300 and one or more other computer systems 1300 or one or more networks. As an example and not by way of limitation, communication interface 1310 may include a network interface controller (NIC) or network adapter for communicating with an Ethernet or other wire-based network or a wireless NIC (WNIC) or wireless adapter for communicating with a wireless network, such as a WI-FI network. This disclosure contemplates any suitable network and any suitable communication interface 1310 for it. As an example and not by way of limitation, computer system 1300 may communicate with an ad hoc network, a personal area network (PAN), a local area network (LAN), a wide area network (WAN), a metropolitan area network (MAN), or one or more portions of the Internet or a combination of two or more of these. One or more portions of one or more of these networks may be wired or wireless. As an example, computer system 1300 may communicate with a wireless PAN (WPAN) (such as, for example, a BLUETOOTH WPAN), a WI-FI network, a WI-MAX network, a cellular telephone network (such as, for example, a Global System for Mobile Communications (GSM) network), or other suitable wireless network or a combination of two or more of these. Computer system 1300 may include any suitable communication interface 1310 for any of these networks, where appropriate. Communication interface 1310 may include one or more communication interfaces 1310, where appropriate. Although this disclosure describes and illustrates a particular communication interface, this disclosure contemplates any suitable communication interface.

In particular embodiments, bus 1312 includes hardware, software, or both coupling components of computer system 1300 to each other. As an example and not by way of limitation, bus 1312 may include an Accelerated Graphics Port (AGP) or other graphics bus, an Enhanced Industry Standard Architecture (EISA) bus, a front-side bus (FSB), a HYPERTRANSPORT (HT) interconnect, an Industry Standard Architecture (ISA) bus, an INFINIBAND interconnect, a low-pin-count (LPC) bus, a memory bus, a Micro Channel Architecture (MCA) bus, a Peripheral Component Interconnect (PCI) bus, a PCI-Express (PCIe) bus, a serial advanced technology attachment (SATA) bus, a Video Electronics Standards Association local (VLB) bus, or another suitable bus or a combination of two or more of these. Bus 1312 may include one or more buses 1312, where appropriate. Although this disclosure describes and illustrates a particular bus, this disclosure contemplates any suitable bus or interconnect.

Herein, a computer-readable non-transitory storage medium or media may include one or more semiconductor-based or other integrated circuits (ICs) (such, as for example, field-programmable gate arrays (FPGAs) or application-specific ICs (ASICs)), hard disk drives (HDDs), hybrid hard drives (HHDs), optical discs, optical disc drives (ODDs), magneto-optical discs, magneto-optical drives, floppy diskettes, floppy disk drives (FDDs), magnetic tapes, solid-state drives (SSDs), RAM-drives, SECURE DIGITAL cards or drives, any other suitable computer-readable non-transitory storage media, or any suitable combination of two or more of these, where appropriate. A computer-readable non-transitory storage medium may be volatile, non-volatile, or a combination of volatile and non-volatile, where appropriate

### 4. Methods of Treatment

The present disclosure relates to methods for preventing and/or treating an aneurysm in a subject.

Aneurysms are excessive localized enlargements of an artery caused by a weakening of the artery wall. The balloon-like bulges have an increased risk of rupture as they increase in size, besides being a potential site for thrombosis and the eventual formation of an embolism. Aneurysms may be the result of a hereditary condition or a later acquired disease. Three particularly lethal types of aneurysms upon rupture are abdominal aortic aneurysm (AAA), thoracic aortic aneurysm (TAA), and cerebral aneurysm (CA). The present disclosure provides methods for preventing and/or treating a cerebral aneurysm.

Cerebral aneurysms (CA) affect about 5 percent of the population and occur when the wall of a blood vessel in the brain becomes weakened and bulges or balloons out. Pre-rupture treatments are generally limited to surgical clipping or endovascular coiling or a flow diverter can be used to seal off an unruptured brain aneurysm and help prevent a future rupture. However, in some unruptured aneurysms, the known risks of the procedures may outweigh the potential benefit.

Cerebral aneurysms are usually found at the base of the brain just inside the skull, in an area called the subarachnoid space. Rupture of these cerebral aneurysms results in bleeding into the space around the brain and is often referred to as subarachnoid hemorrhage (SAH). This kind of hemorrhage can lead to a stroke, coma, and/or death.

Initially, the dysfunctional endothelium secretes neutrophil chemoattractants such as IL-8/CXCL1. Platelets adhere to damaged endothelium, aggregate, and secrete CXCL7 attracting neutrophils via CXCR1/2. The increase in endothelial COX-2 products with the co-current decrease in nitric oxide leads to pro-inflammatory smooth muscle cells, which also attracts macrophages via MCP-1. Neutrophils cause a shift in macrophage phenotype from pro-wound healing M2 phenotype towards pro-inflammatory M1 phenotype. This results in local tissue destruction, aneurysm formation, progression, and eventual rupture. The therapy with GPIIb/IIIa antagonists prevents platelet aggregation, CXCL7 release, and neutrophil infiltration. CXCR1/2 antagonists would allow for a more downstream blockade of effects resulting from platelet activation as well as endothelial cell activation.

In certain non-limiting embodiments, the present disclosure provides for a method of preventing and/or treating aneurysms in a subject. For example, but not by way of limitation, the present disclosure provides a method for preventing and/or treating a cerebral aneurysm in a subject. In certain embodiments, the method can include administering a therapeutically effective amount of an aneurysm inhibitor to the subject.

An “aneurysm inhibitor,” as used herein, can be any molecule, compound, chemical, or composition that has an anti-aneurysm effect and is provided and/or administered in addition to the platelet inhibitors described herein. Aneurysm inhibitors include, but are not limited to, platelet inhibitors, anti-inflammatory, anti-NF-κB inhibitors, calcium channel blockers, protease inhibitors, metalloproteinase inhibitors, mast cell degranulation inhibitors, free radical scavengers, and mineralocorticoid receptor antagonists. Non-limiting examples of secondary aneurysm inhibitors include simvastatin, pravastatin, pitavastatin, valsartan, candesartan, olemsartan, nifedipine, imidapril, ibudilast, celecoxib, tranilast, fasudil, eplerenone, tetracycline, and aspirin. In certain embodiments, the aneurysm inhibitor can be aspirin.

In certain embodiments, administration of the aneurysm inhibitor to the subject has an anti-aneurysm effect or therapeutic benefit. An “anti-aneurysm effect” or “therapeutic benefit” as used herein, refers to one or more of a reduction in aggregate platelet, a reduction in development of an aneurysm, a reduction of growth of an aneurysm, and/or a reduction of rupture of an aneurysm.

In certain embodiments, administration of the aneurysm inhibitor inhibits the development, growth, and/or rupture of an aneurysm in a subject. In certain embodiments, the subject was known to have an aneurysm prior to treatment. In certain non-limiting embodiments, the subject was not known to have an aneurysm prior to treatment.

In certain embodiments, the present disclosure provides methods for reducing the risk of a subject that had an aneurysm from developing new aneurysms, which can include administering a therapeutically effective amount of an aneurysm inhibitor to the subject.

In certain non-limiting embodiments, the present disclosure provides for a method of preventing the growth and rupture of aneurysms, e.g., cerebral aneurysms, in a subject. In certain embodiments, the method includes administering a therapeutically effective amount of an aneurysm inhibitor to the subject. In certain embodiments, preventing an aneurysm includes inhibiting and/or preventing the aggregation of platelets in the endothelium of a subject.

In certain embodiments, an aneurysm inhibitor can be administered to a subject at a dose of about 0.05 mg/kg to about 100 mg/kg. In certain embodiments, a subject can be administered up to about 2,000 mg of the aneurysm inhibitor in a single dose or as a total daily dose. For example, but not by way of limitation, a subject can be administered up to about 1,950 mg, up to about 1,900 mg, up to about 1,850 mg, up to about 1,800 mg, up to about 1,750 mg, up to about 1,700 mg, up to about 1,650 mg, up to about 1,600 mg, up to about 1,550 mg, up to about 1,500 mg, up to about 1,450 mg, up to about 1,400 mg, up to about 1,350 mg, up to about 1,300 mg, up to about 1,250 mg, up to about 1,200 mg, up to about 1,150 mg, up to about 1,100 mg, up to about 1,050 mg, up to about 1,000 mg, up to about 950 mg, up to about 900 mg, up to about 850 mg, up to about 800 mg, up to about 750 mg, up to about 700 mg, up to about 650 mg, up to about 600 mg, up to about 550 mg, up to about 500 mg, up to about 450 mg, up to about 400 mg, up to about 350 mg, up to about 300 mg, up to about 250 mg, up to about 200 mg, up to about 150 mg, up to about 100 mg, up to about 50 mg or up to about 25 mg of the aneurysm inhibitor in a single dose or as a total daily dose. In certain embodiments, the subject can be administered from about 50 to about 1,000 mg of the aneurysm inhibitor in a single dose or a total daily dose. In certain embodiments, a subject can be administered about 1,000 mg of the aneurysm inhibitor, e.g., clopidogrel, in a single dose or as a total daily dose. In certain embodiments, a subject can be administered about 25 mg or more of the aneurysm inhibitor, e.g., clopidogrel, in a single dose or as a total daily dose. In certain embodiments, a subject can be administered about 1,000 mg of the aneurysm inhibitor, e.g., reparixin, in a single dose or as a total daily dose. In certain embodiments, a subject can be administered about 25 mg or more of the aneurysm inhibitor, e.g., reparixin, in a single dose or as a total daily dose.

It is to be understood that, for any particular subject, specific dosage regimes should be adjusted over time according to the individual need and the professional judgment of the person administering or supervising the administration of the aneurysm inhibitor. For example, the dosage of the aneurysm inhibitor can be increased if the lower dose does not provide sufficient activity in the treatment of a disease or condition described herein (e.g., cerebral aneurysm). Alternatively, the dosage of the composition can be decreased if the disease (e.g., cerebral aneurysm) is reduced, no longer detectable, or eliminated.

In certain embodiments, the aneurysm inhibitor can be administered once a day, twice a day, once a week, twice a week, three times a week, four times a week, five times a week, six times a week, once every two weeks, once a month, twice a month, once every other month or once every third month. In certain embodiments, the aneurysm inhibitor can be administered twice a week. In certain embodiments, the aneurysm inhibitor can be administered once a week. In certain embodiments, the aneurysm inhibitor can be administered two times a week for about four weeks and then administered once a week for the remaining duration of the treatment. In certain embodiments, a subject can be administered up to about 1,000 mg of the aneurysm inhibitor in a single dose or as a total daily dose two times a week.

In certain embodiments, the period of treatment can be at least one day, at least one week, at least one month, at least two months, at least three months, at least four months, at least five months, or at least six months. In certain embodiments, the aneurysm inhibitor can be administered until the aneurysm is no longer detectable.

In certain embodiments, the aneurysm inhibitor can be administered to a subject by any route known in the art. In certain embodiments, the aneurysm inhibitor can be administered parenterally. In certain embodiments, the aneurysm inhibitor can be administered orally, intravenously, intraarterially, intrathecally, intranasally, subcutaneously, intramuscularly, and rectally. In certain embodiments, the aneurysm inhibitor can be administered intrathecally. For example, but not by way of limitation, the present disclosure provides methods for the prevention and/or treatment of aneurysm in a subject, e.g., having cerebral aneurysm, by intrathecal administration of an aneurysm inhibitor.

In certain embodiments, one or more aneurysm inhibitors can be used alone or in combination with one or more secondary aneurysm inhibitors. For example, but not by way of limitation, methods of the present disclosure can include administering one or more aneurysm inhibitors. “In combination with,” as used herein, means that the aneurysm inhibitor and a secondary aneurysm inhibitor are administered to a subject as part of a treatment regimen or plan. In certain embodiments, being used in combination does not require that the aneurysm inhibitor and the secondary aneurysm inhibitor are physically combined prior to administration, administered by the same route or that they be administered over the same time frame.

In certain non-limiting embodiments, the present disclosure further provides pharmaceutical formulations of aneurysm inhibitors for therapeutic use. In certain embodiments, the pharmaceutical formulation includes an aneurysm inhibitor and a pharmaceutically acceptable carrier. “Pharmaceutically acceptable,” as used herein, includes any carrier which does not interfere with the effectiveness of the biological activity of the active ingredients, e.g., aneurysm inhibitor, and that is not toxic to the patient to whom it is administered. Non-limiting examples of suitable pharmaceutical carriers include phosphate-buffered saline solutions, water, emulsions, such as oil/water emulsions, various types of wetting agents, and sterile solutions. Additional non-limiting examples of pharmaceutically acceptable carriers can include gels, bioabsorbable matrix materials, implantation elements containing the inhibitor, and/or any other suitable vehicle, delivery, or dispensing means or material. Such carriers can be formulated by conventional methods and can be administered to the subject.

In certain embodiments, the pharmaceutical formulations of the present disclosure include stereoisomers, enantiomers, diastereomers, or racemates of the aneurysm inhibitors. The aneurysm inhibitors disclosed herein can contain one or more asymmetric centers and can thus give rise to enantiomers, diastereomers, and other stereoisomeric forms that can be defined, in terms of absolute stereochemistry, as (R)- or (S)-. In certain embodiments, the pharmaceutical formulation of the present disclosure includes all possible isomers, including racemic mixtures, optically pure forms, and intermediate mixtures. Optically active (R)- and (S)-isomers can be prepared using chiral synthons or chiral reagents or resolved using conventional techniques. If the aneurysm inhibitor contains a double bond, the substituent can be E or Z configuration. If the compound contains a disubstituted cycloalkyl, the cycloalkyl substituent can have a cis- or trans-configuration. All tautomeric forms are also intended to be included.

In certain embodiments, the pharmaceutical formulations of the present disclosure can be formulated using pharmaceutically acceptable carriers well known in the art that are suitable for parenteral administration, e.g., intravenous administration, intraarterial administration, intrathecal administration, intranasal administration, intramuscular administration, subcutaneous administration, and intracisternal administration. In certain embodiments, the pharmaceutical formulation is formulated for intrathecal administration. For example, but not by way of limitation, the pharmaceutical formulation can be formulated as solutions, suspensions, or emulsions.

In certain non-limiting embodiments, the pharmaceutical formulations of the present disclosure can be formulated using pharmaceutically acceptable carriers well known in the art that are suitable for oral administration. Such carriers enable the pharmaceutical compositions to be formulated as tablets, pills, capsules, liquids, gels, syrups, slurries, suspensions, and the like, for oral ingestion by a patient to be treated. In certain embodiments, the pharmaceutical formulation can be a solid dosage form.

In certain embodiments, the pharmaceutical formulation can be formulated to release the aneurysm inhibitor immediately upon administration. Alternatively, the pharmaceutical formulation can be formulated to release the aneurysm inhibitor at any predetermined time or time period after administration. Such types of compositions are generally known as controlled release formulations, which include (i) formulations that create substantially constant concentrations of the aneurysm inhibitor within the subject over an extended period of time; (ii) formulations that after a predetermined lag time create substantially constant concentrations of the aneurysm inhibitor within the subject over an extended period of time; (iii) formulations that sustain the aneurysm inhibitor's action during a predetermined time period by maintaining a relatively constant, effective level of the aneurysm inhibitor in the body with concomitant minimization of undesirable side effects; (iv) formulations that localize action of aneurysm inhibitor, e.g., spatial placement of a controlled release composition adjacent to or in the disease, e.g., endothelial cells, platelet cells; (v) formulations that achieve convenience of dosing, e.g., administering the composition once per week or once every two weeks; and (vi) formulations that target the action of the aneurysm inhibitor by using carriers or chemical derivatives to deliver the aneurysm inhibitor to a particular target cell type or a particular target tissue type. In certain embodiments, controlled release is obtained by appropriate selection of various formulation parameters and ingredients, including, e.g., various types of controlled release compositions and coatings. For example, but not by way of limitation, the aneurysm inhibitor can be formulated with appropriate excipients into a pharmaceutical formulation that, upon administration, releases the aneurysm inhibitor in a controlled manner, e.g., oil solutions, suspensions, emulsions, microcapsules, molecular complexes, microspheres, nanoparticles, patches, and liposomes.

In certain embodiments, the pharmaceutical formulations suitable for use in the present disclosure can include formulations where the aneurysm inhibitors are contained in a therapeutically effective amount. A “therapeutically effective amount” refers to an amount that is able to prevent and/or reduce the development, growth, and rupture of an aneurysm. The therapeutically effective amount of an active ingredient can vary depending on the active ingredient, e.g., aneurysm inhibitor, formulation used, the anatomical location of the aneurysm and its severity, and the age, weight, etc., of the subject to be treated. In certain embodiments, a patient can receive a therapeutically effective amount of an aneurysm inhibitor as a single dose or multiple administrations of two or more doses, which can depend on the dosage and frequency as required and tolerated by the patient. In certain embodiments, the provided methods involve administering the compositions at effective amounts, e.g., therapeutically effective amounts.

In certain non-limiting embodiments, the present disclosure provides a method of treating a subject having an aneurysm that includes diagnosing aneurysm in the subject and then treating the subject with an aneurysm inhibitor. In certain embodiments, the method for diagnosing aneurysms includes determining the levels of a biomarker, as disclosed in Section 2 above.

In certain embodiments, the method for diagnosing aneurysms includes performing magnetic resonance imaging (MRI) of the brain or abdomen, magnetic resonance angiography (MRA), computed tomography angiography scan (CTA scan), angiogram, or cerebrospinal fluid test. Additional methods for diagnosing aneurysm are disclosed in Calero and Illig, Semin Vasc Surg. 2016; 29(1-2):3-17, the contents of which are incorporated by reference herein.

### 4.1. Platelet Inhibitors

In certain embodiments, the present disclosure provides methods for preventing and/or treating an aneurysm in a subject by inhibiting the platelet activation and aggregation in the subject. In certain embodiments, the aneurysm inhibitor is a platelet inhibitor. A platelet inhibitor can be a molecule, e.g., chemical compound, that inhibits the process of platelet formation. A platelet inhibitor can be a molecule, e.g., chemical compound, that inhibits the process of platelet activation. A platelet inhibitor can be a molecule, e.g., chemical compound, that inhibits thrombus formation. A platelet inhibitor can reversibly or irreversibly inhibit the process involved in platelet activation resulting in decreased tendency of platelets to adhere to one another and to damaged blood vessels' endothelium.

Non-limiting examples of platelet inhibitors for use in the present disclosure include irreversible cyclooxygenase inhibitors (e.g., aspirin), adenosine diphosphate (ADP) receptor inhibitors (e.g., ticlopidine), phosphodiesterase inhibitors (e.g., vorapaxar), inhibitors of glycoprotein IIB/IIIA (e.g., abciximab), adenosine reuptake inhibitors, thromboxane inhibitors, thromboxane synthase inhibitors, thromboxane receptor antagonists, terutroban, salts thereof, or derivatives thereof. Additional examples of platelet inhibitors for use in the present disclosure include, without any limitation, interfering ribonucleic acids (e.g., siRNA, shRNA), antibodies, aptamers, or peptidomimetics.

In certain embodiments, the platelet inhibitor for use in the present disclosure is an inhibitor of glycoprotein IIB/IIIA (GPIIB/IIIA). GPIIB/IIIA is a receptor on the platelet surface that undergoes a conformational change upon activation of the platelet allowing it to bind plasma fibrinogen. Because multiple GPIIB/IIIA molecules from different platelets can bind the same fibrinogen molecule, this facilitates platelet aggregation at sites of vascular injury. By preventing the GPIIB/IIIA molecule from interacting with fibrinogen these inhibitors consequently interfere with the process of platelet aggregation.

Non-limiting examples of inhibitors of glycoprotein IIB/IIIA for use in the present disclosure include abciximab, eptifibatide, tirofiban, lefradafiban, fredabin, lamifiban, clopidogrel, orbofiban, roxifiban, sibrafiban, xemilofiban, ticlopidine, ticagrelor, prasugrel, LM-609, resveratrol, ferric cation, levothyroxine, YM-57029, YM128, a non-peptide mimetic of the tetrapeptide RGDF, a peptide mimetic of the tetrapeptide RGDF, salts thereof, or derivatives thereof. In certain embodiments, the inhibitor of glycoprotein IIB/IIIA is clopidogrel, a salt thereof or a derivative thereof.

In certain embodiments, the inhibitor of glycoprotein IIB/IIIA has the following formula:

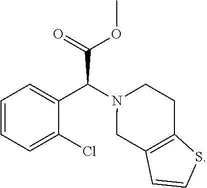 [SMILES: COC(=O)[C@H](c1ccccc1Cl)N1CCc2sccc2C1]

In certain embodiments, the platelet inhibitor for use in the present disclosure inhibits the platelet-driven CXCL7-CXCR1/2 pathway. CXCL7 is a small cytokine belonging to the chemokine family and binds CXCR1 and CXCR2 receptors. CXCL7 exerts its function by activating the CXCR1 and/or CXCR2 and binding sulfated glycosaminoglycans (GAGs) that regulate receptor activity and is released by platelets upon their activation. Upon activation, both CXCR1 and CXCR2 transfer the signal into the cell which results in platelet dysfunction and aneurysm growth. By preventing the interaction of CXCL7 with its receptors and by inhibiting the activity of the CXCR1 and/or CXCR2, these inhibitors consequently interfere with the process of aneurysm development and growth.

In certain embodiments, the platelet inhibitor for use in the present disclosure is an inhibitor of the chemokine (C-X-C motif) ligand 7 (CXCL7). In certain embodiments, the inhibitor of CXCL7 is ethanesulfonic acid. In certain embodiments, the inhibitor of CXCL7 is an antibody anti-CXCL7, or a fragment thereof. In certain embodiments, the antibody can be monoclonal. In certain embodiments, the antibody can be polyclonal. In certain embodiments, the antibody can be humanized. Non-limiting examples of antibodies anti-CXCL7 are disclosed in International Patent Application Nos. PCT/US2011/024123 and PCT/EP2014/060201, which are incorporated herein by reference in their entireties.

In certain embodiments, the platelet inhibitor for use in the present disclosure is an inhibitor of the C-X-C chemokine receptor type 1 (CXCR1). In certain embodiments, the platelet inhibitor for use in the present disclosure is an inhibitor of the C-X-C chemokine receptor type 2 (CXCR2). In certain embodiments, the platelet inhibitor for use in the present disclosure is an inhibitor of CXCR1 and CXCR2. Non-limiting examples of inhibitors of CXCR1 and CXCR2 for use in the present disclosure include SX-682, AZD5069, AZD8797, QBM076, reparixin, SCH-527123, danirixin, navarixin, ladarixin, SB225002, nicotinamide N-oxide, UNBS5162, CXCR2-IN-1, SRT3109, SCH563705, SRT3190, SB265610, elubrixin, SB332235, carydalmine, salts thereof or derivatives thereof. In certain embodiments, the inhibitor of CXCR1 and CXCR2 is reparixin, a salt thereof, or a derivative thereof. In certain embodiments, the inhibitor of CXCR1 and CXCR2 has the following formula:

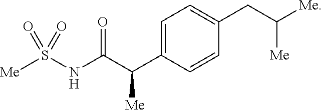 [SMILES: CC(C)Cc1ccc([C@@H](C)C(=O)NS(C)(=O)=O)cc1]

In certain embodiments, the platelet inhibitor for use in the present disclosure is a nucleic acid targeting a protein regulating the platelet-driven CXCL7-CXCR1/2 pathway. In certain embodiments, the nucleic acid targets CXCL7. In certain embodiments, the nucleic acid targets CXCR1 receptor. In certain embodiments, the nucleic acid targets CXCR2 receptor. Non-limiting examples of nucleic acids for use in the present disclosure include siRNAs and shRNAs. siRNA molecules are polynucleotides that are generally about 20 to about 25 nucleotides long and are designed to bind specific RNA sequence (e.g., CXCR1 mRNA or CXCR2 mRNA). siRNAs silence gene expression in a sequence-specific manner, binding to a target RNA (e.g., an RNA having the complementary sequence) and causing the RNA to be degraded by endoribonucleases. siRNA molecules able to inhibit the expression of the CXCR1 or CXCR2 can be produced by suitable methods. There are several algorithms that can be used to design siRNA molecules that bind the sequence of a gene of interest (see e.g., Huesken et al., Nat. Biotechnol. 23:995-1001; Jagla et al., RNA 11:864-872, 2005; Shabalinea, BMC Bioinformatics 7:65, 2005). Additionally or alternatively, expression vectors expressing siRNA or shRNA can be used (see e.g., Brummelkamp, Science 296: 550-553, 2002; Lee et al., Nature Biotechnol. 20:500-505, 2002; Elbashir et al., Nature 411:494-498, 2001).

In certain embodiments, the platelet inhibitor for use in the present disclosure is a ribozyme that inhibits the expression of CXCR1 and/or CXCR2. Ribozymes are RNA molecules possessing enzymatic activity. One class of ribozymes is capable of repeatedly cleaving other separate RNA molecules into two or more pieces in a nucleotide base sequence specific manner (see Kim et al., Proc Natl Acad Sci USA, 84:8788 (1987); Haseloff & Gerlach, Nature, 334:585 (1988); and Jefferies et al., Nucleic Acid Res, 17:1371 (1989). Such ribozymes typically have two functional domains: a catalytic domain and a binding sequence that guides the binding of ribozymes to a target RNA through complementary base-pairing. Once a specifically-designed ribozyme is bound to a target mRNA, it enzymatically cleaves the target mRNA, reducing its stability and destroying its ability to directly translate an encoded protein. Methods for selecting a ribozyme target sequence and designing and making ribozymes are generally known in the art.

In certain embodiments, the platelet inhibitor for use in the present disclosure is a gene-editing system that inhibits the expression of CXCR1 and/or CXCR2. Non-limiting examples of gene-editing systems for use in the present disclosure include transcription activator-like effector nucleases (TALENs), zinc-finger nucleases, meganuclease, clustered regularly interspaced short palindromic repeat-associated proteins (CRISPR/Cas9), DNA-repair proteins, DNA-modification proteins, and DNA methyltransferases. Details on the gene-editing systems for use in the present disclosure can be found in Adli et al., Nat Commun. 2018 May 15; 9(1):1911 and Maeder & Gersbach, Mol Ther. 2016 March; 24(3):430-46, the content of each of which is incorporated by reference in its entirety.

### 5. Kits

The present disclosure provides kits for treating a subject having or suspected to have an aneurysm. In certain embodiments, the kits include an effective amount of an aneurysm inhibitor or a pharmaceutical composition including said inhibitor in unit dosage form. Non-limiting exemplary of said aneurysm inhibitors and methods of use can be found in Section 3. In certain embodiments, the kits include a sterile container that contains the agents or the genetic engineering system; such containers can be boxes, ampules, bottles, vials, tubes, bags, pouches, blister-packs, or other suitable container forms known in the art. Such containers can be made of plastic, glass, laminated paper, metal foil, or other materials suitable for holding medicaments.

In certain embodiments, the kits include instructions for administering an aneurysm inhibitor to a subject having or suspected to have an aneurysm. The instructions can include information about the use of the aneurysm inhibitor or pharmaceutical composition for treating the aneurysm. In certain embodiments, the instructions include at least one of the following: description of the aneurysm inhibitor; dosage schedule and administration for treating the aneurysm; precautions; warnings; indications; counter-indications; over dosage information; adverse reactions; animal pharmacology; clinical studies; and/or references. The instructions can be printed directly on the container (when present), or as a label applied to the container, or as a separate sheet, pamphlet, card, or folder supplied in or with the container.

The present disclosure further provides kits for diagnosing an aneurysm. The present disclosure further provides for diagnosing the rupture of an aneurysm. In certain embodiments, the kits are configured for detecting a level of a biomarker, e.g., using a detector. Non-limiting exemplary of said biomarkers and methods can be found in Section 2.

Non-limiting examples of detectors that can be used with the presently disclosed kits include antibodies for immunodetection of the biomarker to be identified, oligonucleotide primers suitable for polymerase chain reaction (PCR), or nucleic acid sequencing; nucleic acid probes suitable for in situ hybridization or fluorescent in situ hybridization.

In certain embodiments, the kit further includes instructions or supporting material that describe the use of the kit to diagnose an aneurysm and/or reference to a website or publication describing the same. In certain embodiments, the kit further includes instructions or supporting material that describe the use of the kit to diagnose the risk of rupture of an aneurysm and/or reference to a website or publication describing the same.

### EXAMPLES

The presently disclosed subject matter will be better understood by reference to the following Examples, which are provided as exemplary of the presently disclosed subject matter, and not by way of limitation.

### Example 1. Blockade of the Platelet-Driven CXCL7-CXCR1/2 Inflammatory Pathway

The present example shows that the platelet-driven CXCL7-CXCR1/2 inflammation pathway is involved in aneurysm formation. The present example demonstrated in a murine cerebral aneurysm model that pharmacological blockade of the CXCL7-CXCR1/2 pathway significantly reduced aneurysm formation. Mice treated with 1 mg/kg clopidogrel developed significantly less aneurysms than controls (0% vs 57%, n=6 and 7 respectively, p<0.001). The CXCL7-CXCR1/2 axis was targeted in mice using 10 mg/kg reparixin (CXCR1/2 antagonist) and resulted in decreased aneurysm formation (14% vs 57%, n=6 and 7, p=0.0163).

Intracranial arteries are composed of endothelial and vascular smooth muscle cells (VSMCs) and a layer of elastin between the two known as the internal elastic lamina. Shear stress is a force that is experienced at a boundary parallel to the direction of the fluid flow and is particularly important for the health of blood vessels. Aneurysms are thought to form through an inflammatory mediated hemodynamic process at areas where shear stress is initially high. Then, they continue to grow in dome regions where shear stress is much lower than in the surrounding environment. IL-8/CXCL1 secreted by endothelial cells is critical in early cerebral aneurysm formation process. This early process is primarily dominated by neutrophil infiltration. These low flow conditions set the stage for platelet aggregation, which can lead to further inflammation.

There is a critical role of CXCR1/2 ligands and platelet activation in cerebral aneurysm formation. IL-8 is a chemokine that attracts neutrophils to sites of inflammation-causing local remodeling. IL-8 and CXCL1 are two of five different cytokines capable of activating receptors CXCR1/2. Platelets are a nuclear cell elements having a primary role in hemostasis. Activated platelets are capable of secreting C-X-C ligand-7 (CXCL7), which binds to CXCR1/2 and leads to further neutrophil infiltration. Thrombosis has been shown to promote infiltration of leukocytes into inflamed tissues through a chemotactic gradient mediated by CXCL7 and receptors CXCR1/2. The inflammatory cascade in cerebral aneurysm pathophysiology is multifaceted. Platelet-neutrophil aggregate formation leads to co-current activation and increased inflammatory response with extravasation into local vascular environment. These platelet-neutrophil complexes have been found to aggravate atherosclerosis through a positive feedback loop involving CXCL1, CXCR1/2, and CXCL7. Aneurysms continue to grow through specific inflammatory stages: 1) platelet aggregation and activation, 2) platelet-induced inflammatory response, and 3) pro-inflammatory M1 macrophage formation, resulting in 4) rupture.

### Example 2. Murine Cytokine Analysis and Development of Inflammatory Signature Model

The present example shows that the murine cerebral aneurysm micro-environment displayed specific cytokine profiles at different time points and with treatment. Semi-quantitative analysis of 96 different cytokines showed increased protein expression of CXCL7 in murine cerebral aneurysms when compared to controls. The CXCL7-CXCR1/2 pathway in mice was implicated in platelet inflammation.

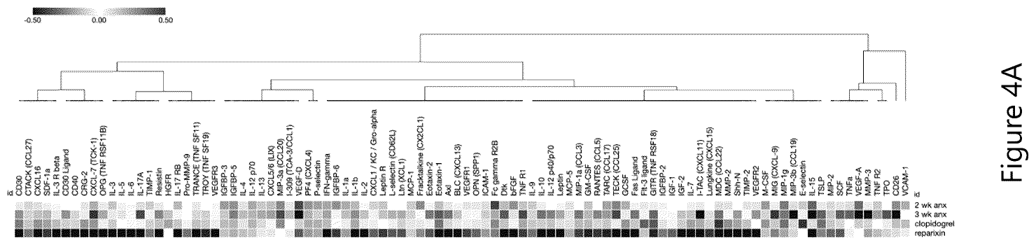

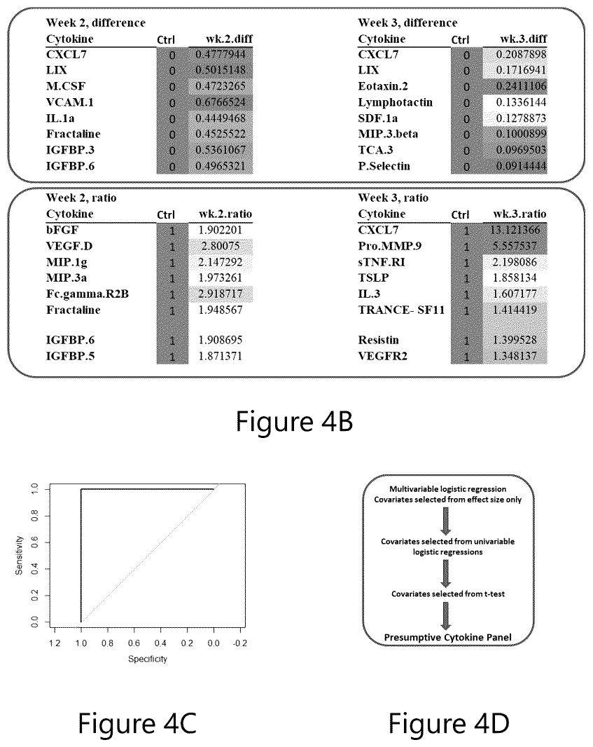

Temporal analysis and comparison with pharmacologic treatment were performed. An analysis of 96 different cytokines showed differential inflammatory protein expression at 2 weeks and there was gradual decline of most cytokines at 3 weeks. Increased cytokine expression was noted for CXCL7, IL-17, and the NF-kB pathway across all timepoints. The levels of IL-8 murine homolog CXCL1, monokine induced by gamma (MIG) and macrophage inflammatory protein (MIP) families had a robust response at 2 weeks that then declined at 3 weeks. Blockade of CXCR1/2 receptors using reparixin resulted in vastly attenuated inflammatory profile when compared to PBS-treated aneurysmal mice or control sham surgery animals (FIG. 4A).

An inflammatory fingerprint signature for murine cerebral aneurysms was observed at 2 and 3 weeks (FIG. 4A) by selecting eight (8) differentially expressed cytokines via either absolute levels or relative ratio (FIG. 4B). Two models, developed to derive aneurysm predictions, were then created by the “Effects size” or “Logistic Regression” approach. These models were used to predict presence of cerebral aneurysm in known murine samples in a blinded fashion. Before feeding data into the models for prediction, samples were de-identified by innovator KWN resulting in a test with 100% sensitivity and 100% specificity (FIG. 4C). This approach was then used to develop a 6-cytokine presumptive human-equivalent mini-cytokine panel as a diagnostic test to detect cerebral aneurysm formation (FIG. 5D).

The developed mini-panel test to cytokine array data obtained from mouse cervical carotid aneurysms showed that the test was able to differentiate between intracranial and cervical carotid aneurysms. Notably, the test did not falsely identify cervical carotid aneurysms as intracranial aneurysms.

### Example 3. Cytokine Panel for Predicting Aneurysms in Humans

The present example shows cytokine expression profiling detected and differentiated aneurysms in humans. The present example used previously collected, de-identified peripheral blood, aneurysm dome, and superficial temporal artery samples from the IRB-approved Thunda-DOME tissue-bank, which contained paired specimens from 19 unique patients (FIGS. 5A-5D). Location of aneurysms within Circle of Willis from the human tissue bank did not show any unusual patterns (n=21, paired human aneurysm dome, STA tissue, and venous blood samples 14 female, 7 male, 2 ruptured, 19 unruptured, 9 smokers, 4 non-smokers, 8 unknown status) (FIG. 5A). Analysis of peripheral blood samples from patients with aneurysms also did not show any unusual patterns (FIG. 5A). There were no significant differences in white blood cell (WBC) count, immune cell counts, or platelet counts. Semi-quantitative arrays showed that several cytokines of interest out of a panel of 120 cytokines were consistently and differentially increased in peripheral blood samples of patients with unruptured cerebral aneurysms (n=19) and ruptured cerebral aneurysms (n=2), when compared to presumed healthy controls (n=3), meningioma patients (n=5), metastasis patients (n=3), and patients harboring glioblastoma multiforme (n=4, data not shown) (FIG. 5A). Meanwhile, aneurysm dome tissue samples showed generalized overall increased expression of almost all inflammatory markers (FIG. 5A).

Human inflammatory profiles or fingerprint models were developed based on the murine model described in Example 2 and human cytokine arrays shown in FIG. 5A. An initial preliminary two-cytokine model was developed to predict and differentiate between aneurysmal samples and reference serum with 81.0% sensitivity and 75.0% specificity (FIG. 5B). The assay included a balanced model with respect to sensitivity and specificity (FIG. 5C) and a high sensitivity model (FIG. 5D). Application of the 6-cytokine panel tests, using the least absolute shrinkage and selection operator method (LASSO method), predicted and differentiated between aneurysmal samples and reference serum with 95.2% sensitivity and 90.9% specificity using the balanced model (FIG. 5C), and 100% sensitivity and 90.9% specificity using the high sensitivity model (FIG. 5D) (blinded) (peripheral blood n=21, aneurysm dome n=3, reference serum n=3, total control=11).

To address the potential aneurysm rupture, the 6-cytokine panel test, using a “Logistic Regression” approach, was used to predict and differentiate between ruptured aneurysmal samples and unruptured aneurysmal samples with 100% sensitivity and 100% specificity (blinded) (peripheral blood of unruptured patients n=19, peripheral blood of ruptured patients n=2) (FIG. 5E).

The present example also shows that the above-mentioned models can further used to define inflammatory signature sub-groups between those harboring aneurysms. FIG. 8A shows that a simple application of a multi-cytokine panel (in this case a panel with 120 different cytokines) does not distinguish peripheral blood samples from patients with aneurysms when compared to reference controls. However, FIG. 8B shows a subgroup analysis (principal component analysis) after application of the model shown in FIG. 5C is able to differentiate peripheral blood samples from patients harboring aneurysm(s) from reference controls. There were four distinct inflammatory subgroups identified in patients harboring known aneurysms (groups 2-5), compared to reference controls (group 0). Only one group of undefined aneurysms did not harbor other differentiating inflammatory markers (group 1). Further subgroup cerebral aneurysms were used to evaluate the progression of unruptured cerebral aneurysms.

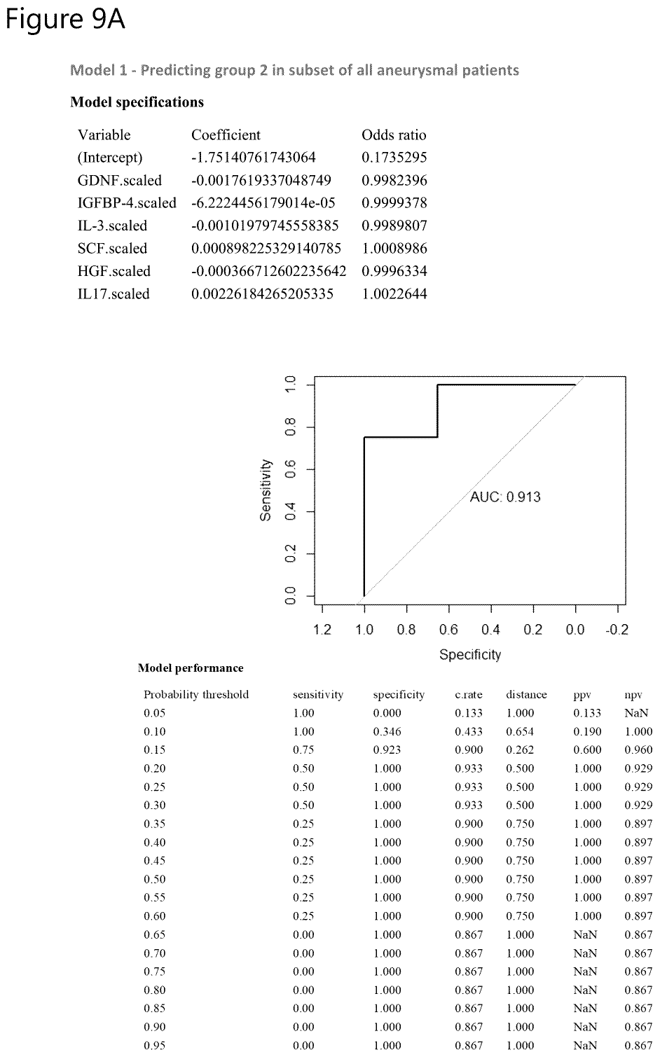

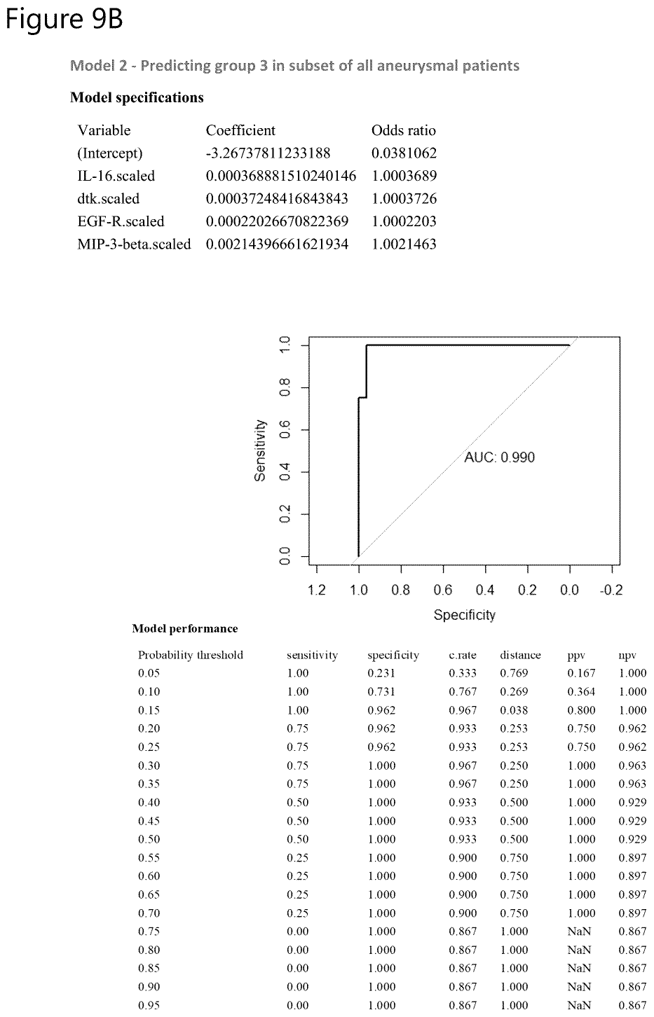

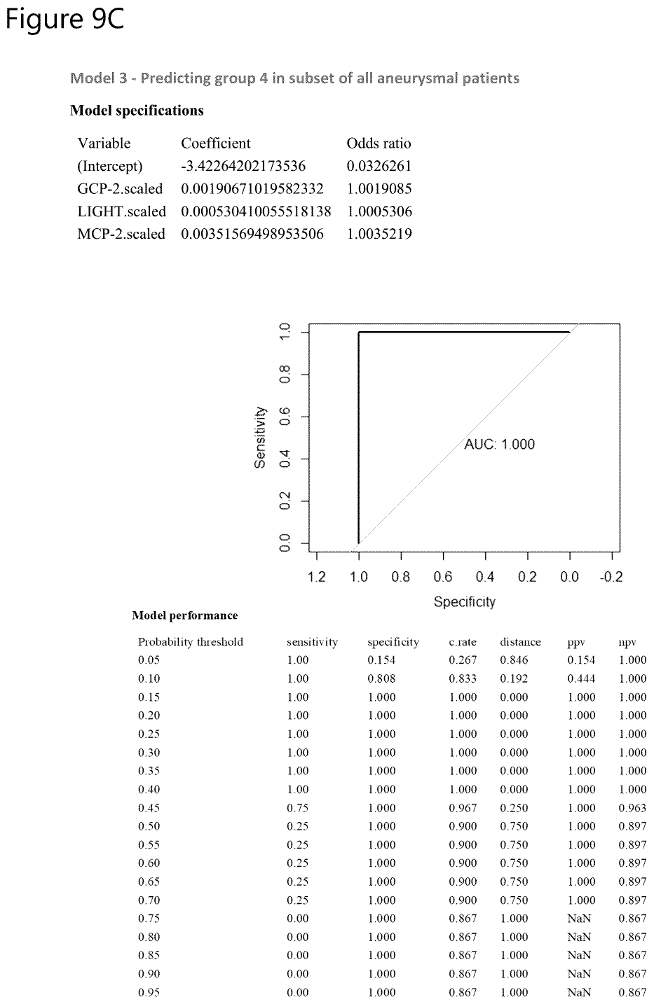

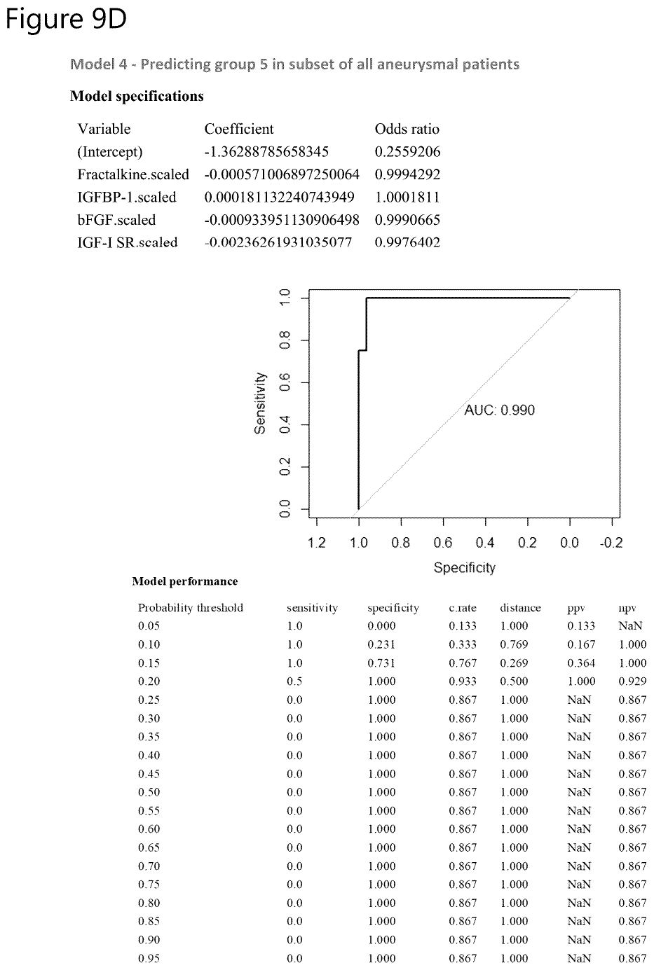

FIGS. 9A-9D show the application of a subsequent 6-cytokine panel test derived from the human cytokine arrays (FIG. 5A) predict and differentiate between various subgroups and other groups within the peripheral blood of patients harboring aneurysms.

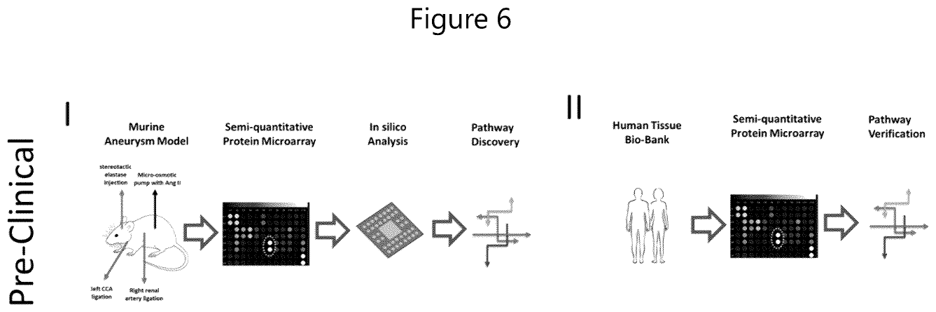

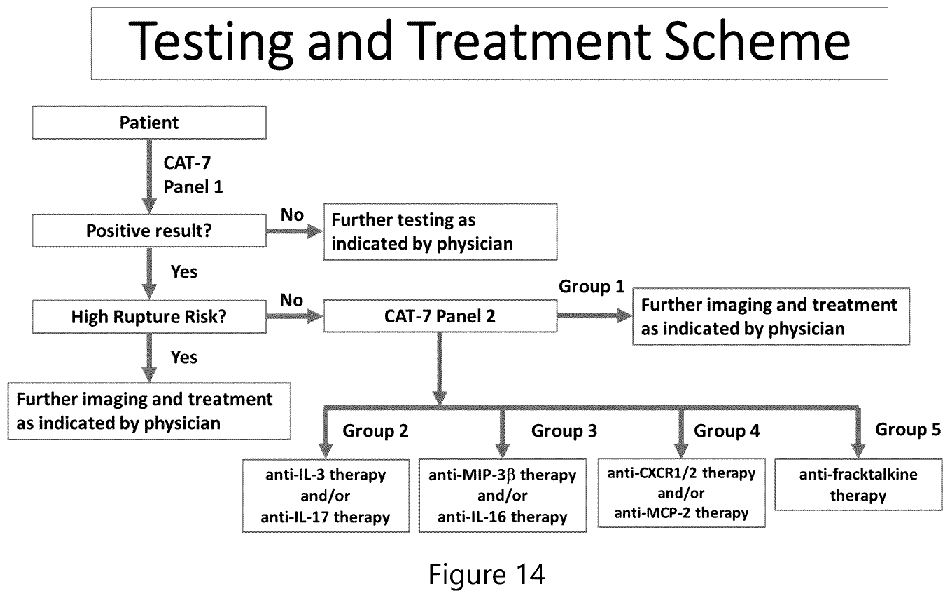

The present example shows the development of a blood test for cerebral aneurysm detection based on a pre-clinical vertebrate aneurysm model and previously collected, de-identified human blood and cerebral aneurysm samples (FIG. 6). Additionally, the blood test can be used to determine a whether a patient belongs to a specific inflammatory subgroup and provide an appropriate anti-inflammatory treatment strategy (FIG. 14). The present example shows a simplified approach that involved determining the specific temporal “fingerprint” signature of cerebral aneurysms of different chronicity within an animal model to develop a combination mini-cytokine panel for detection of cerebral aneurysms in the human general population. The developed mini-panel provides a superior test able to accurately pick out inflammatory signatures of aneurysms at different stages of the disease.

### Example 4. Development of Diagnostic Test

The present example shows the development of a diagnostic test used to determine the probability of a patient harboring an aneurysm as well as their risk of potential catastrophic rupture. Additionally, the diagnostic test can be used to identify distinct inflammatory subgroups of cerebral aneurysms. Building a diagnostic test required data extraction, model building, probability calculation, and thresholding. The diagnostic test used to determine the probability of a patient harboring an aneurysm to was developed based on murine inflammatory signature profiles or fingerprint models (Example 1) and human cytokine array data (Example 3). To differentiate between rupture and unruptured aneurysm an interactive approach with retrospective human cytokine data (FIG. 7) and sample stratification based on t-SNE inflammatory cytokine analysis (FIGS. 8A and 8B) was applied as shown in FIGS. 10A and 10B.

The first model developed, based on data collected from mice sacrificed at 2 weeks, predicts the likelihood of aneurysm presence (Equation 1, Table 1, and Equation 4). Tables 2 and 3 show examples of values used to arrive at prediction from calculations obtained from Equation 4.

### Equation 1. General Form of Probability Equation:

$$P = \frac{{e}^{{β}_{0} + {β}_{1} {X}_{1} ⋯ + {β}_{n} {X}_{n}}}{1 + {e}^{{β}_{0} + {β}_{1} {X}_{1} ⋯ + {β}_{n} {X}_{n}}}$$

Where P is the probability of the event, Po is the value of the intercept from the model, β₁ is the coefficient of the first covariate from the model, and X₁ is the value of the first covariate in the experimental data. This equation extends for as many covariates as are included in the model.

TABLE 1
Values of intercept and coefficient in model derived
from pre-clinical Week 2 data using covariates selected
from univariable logistic regression.

| Coefficient | Value |
|---|---|
| Intercept | −1.2123520 |
| Fas Ligand | 0.0005358 |
| MDC/CCL22 | 0.0006682 |

Equation 4. Specific Form of the Equation in Model Generated from Week 2 Data Using Covariates

$$P = \frac{{e}^{- 1.212352 + ( 0.0005358 \times Fas . Ligand ) + ( 0.0006682 \times MDC .. CCL 22 )}}{1 + {e}^{- 1.212352 + ( 0.0005358 \times Fas . Ligand ) + ( 0.0006682 \times MDC .. CCL 22 )}}$$

To generate a prediction, insert values of each variable from the experimental data and solve the equation.

TABLE 2
Cytokine concentrations from Subject “foxtrot”
in experimental data.

| Cytokine | Value |
|---|---|
| Fas Ligand | 204.2 |
| MDC/CCL22 | 193.5 |

Inserting these values into Equation 4 yields a probability of 0.999 that Subject “foxtrot” harbors an aneurysm, a near certainty. For a binary prediction the probability threshold is defined as 0.5, above which predicted presence of an aneurysm and below which predicted absence of an aneurysm. In this case, it would be predicted that this individual does harbor an aneurysm. Subject “foxtrot” does harbor an aneurysm and the prediction was correct.

TABLE 3
Cytokine concentrations from Subject “zeta” in experimental data.

| Cytokine | Value |
|---|---|
| Fas Ligand | 169.8 |
| MDC/CCL22 | 43.1 |

Inserting these values into Equation 4 yields a probability of 0.001 that Subject “zeta” does not harbor an aneurysm, a near certainty. Employing the same probability threshold of 0.5 predicts absence of an aneurysm. Subject “zeta” does not, in fact, harbor an aneurysm and the prediction is correct. The second model is used to predict likelihood of ruptured aneurysm or impending rupture (Table 4 and Equation 5).

TABLE 4
Values of intercept and coefficient in model derived from Week 3 data
using covariates selected from univariable logistic regression.

| Coefficient | Value |
|---|---|
| Intercept | 120.8023 |
| RANTES. | 0.1731 |
| IL-12 p40/p70 | −3.5717 |
| MIP-1a | 1.0532 |
| sTNF RI | 2.6095 |
| MCP-1 | −0.5535 |

Equation 5. Specific Form of the Equation in Model Generated from Week 3 Data Using Covariates Selected from Univariable Logistic Regression (Coefficients Rounded to 1 Decimal Point):

$$P = \frac{{e}^{120.8 + ( 0.17 \times RANTES ) + ( - 3.6 \times IL \text{.12} . p 40. p 70 ) + ( 1.1 \times MIP \text{.1} a ) + ( 2.6 \times sTNF . RI ) + ( - 0.6 \times MCP \text{.1} )}}{1 + {e}^{120.8 + ( 0.17 \times RANTES ) + ( - 3.6 \times IL \text{.12} . p 40. p 70 ) + ( 1.1 \times MIP \text{.1} a ) + ( 2.6 \times sTNF . RI ) + ( - 0.6 \times MCP \text{.1} )}}$$

Tables 5 and 6 show examples of values used to arrive at prediction from calculations obtained from Equation 5.

TABLE 5
Cytokine concentrations from Subject “foxtrot”
in experimental data.

| Cytokine | Value |
|---|---|
| RANTES. | 1587.0673 |
| IL-12 p40/p70 | 101.8735 |
| MIP-1a | 75.81023 |
| sTNF-RI | 302.1077 |
| MCP-1 | 1195.3549 |

Inserting these values into Equation 5 yields a probability of 0.999 that Subject “foxtrot” harbors a ruptured aneurysm or an aneurysm with impending rupture. Using a probability threshold of 0.5, the model predicts that Subject “foxtrot” harbors a ruptured aneurysm which is correct.

In a clinical setting, this model would be used to predict risk of rupture if an aneurysm is detected by the first model.

TABLE 6
Cytokine concentrations from Subject “zeta” in experimental data

| Cytokine | Value |
|---|---|
| RANTES. | 1898.4730 |
| IL-12 p40/p70 | 836.3690 |
| MIP-1a | 387.4248 |
| sTNF RI | 925.7370 |
| MCP-1 | 1051.1652 |

Inserting these values into Equation 5 yields a probability of 0.001 that Subject “zeta” does not harbor a ruptured aneurysm or aneurysm with impending rupture. Applying the probability threshold of 0.5 predicts that this individual does not harbor a ruptured aneurysm which is a correct prediction.

In a true clinical scenario, the second model would not be used to predict impending aneurysmal rupture since the subject was not predicted to have an aneurysm by the first model. However, the calculations are shown for completion.

Tables 7 and 8 show sample calculations, probability results, and predictions of model 1 (Equation 4) and model 2 (Equation 5) after detection threshold optimization via training and validation cohorts. Samples from the human retrospective aneurysm tissue bank were evenly and randomly assigned to either training or validation cohort.

TABLE 7
Model 1 predicting aneurysm presence based on human data alone.

| | | | Aneurysm | |
|---|---|---|---|---|
| Subject | Fas Ligand | MDC/CCL22 | (yes/no) | Probability |
| Odds ratio | 1.022 | 1.007 | | |
| 21 (ruptured) | 204.200363 | 193.509583 | 1 | 0.796 |
| 8 (unruptured) | 563.7477 | 401.94364 | 1 | 1 |
| Sierra | 120.52064 | 118.16259 | 0 | 0.284 |
| (ref serum) | | | | |

TABLE 8
Model 2 predicting aneurysm rupture based on human data alone.

| | | IL-12 | | | | Rupture | |
|---|---|---|---|---|---|---|---|
| Subject | RANTES | p40p70 | MIP-1a | sTNF RI | MCP-1 | (yes/no) | Probability |
| Odds ratio | 0.954 | 1.199 | 1.103 | 0.742 | 1.09 | | |
| 10 (ruptured) | 1653.06118 | 323.07365 | 135.94532 | 305.34965 | 586.75248 | 1 | 1 |
| 8 (unruptured) | 2677.28853 | 736.96936 | 585.35711 | 1311.80904 | 74.66475 | 0 | 0 |

Human data was used to optimize detection thresholds in the final diagnostic test model. The first cohort is comprised only of subjects with aneurysms. The training set is made up of 5 subjects without rupture and 1 with rupture. The validation set is made up of 1 subject with and 1 without rupture. The model perfectly classified the two subjects on whom it was tested.

Two additional models were developed to predict the likelihood of an aneurysm presence. However, rather than 2-cytokine panel based approach, these models were developed using a panel of 6-cytokine biomarkers. One model represents a balanced approach with respect to sensitivity and specificity (Table 9 and FIG. 5C), while another model is one of high sensitivity (Table 10 and FIG. 5D).

TABLE 9
Values of intercept, coefficient, and odds ratio in a balanced
model from data of a 6-cytokine panel using the least absolute
shrinkage and selection operator method (LASSO method).

| Variable | Coefficient | Odds Ratio |
|---|---|---|
| Intercept | −0.524441133315259 | 0.5918861 |
| I-309.scaled | 0.00123013121603709 | 1.0012309 |
| IL-16.scaled | 0.000860277388588843 | 1.0008606 |
| IL-1alpha.scaled | −4.51081677896827e−05 | 0.999549 |
| MCP-2.scaled | 0.000352183613428327 | 1.0003522 |
| MIP-1-delta.scaled | 0.00102749322758205 | 1.001028 |
| uPAR.scaled | 0.000110686985879538 | 1.0001107 |

TABLE 10
Values of intercept, coefficient, and odds ratio in a high sensitivity
model from data of a 6-cytokine panel using the least absolute
shrinkage and selection operator method (LASSO method).

| Variable | Coefficient | Odds Ratio |
|---|---|---|
| Intercept | −0.307187663385877 | 0.7355126 |
| I-309.scaled | 0.00174765438331817 | 1.0017492 |
| IL-16.scaled | 0.00102301439669183 | 1.0010235 |
| MCP-4.scaled | 4.4909487563449e−07 | 1.0000004 |
| MIP-1-delta.scaled | 0.000371583141415427 | 1.0003717 |
| CXL7/NAP-2.scaled | 0.000346312839791164 | 1.0003464 |
| uPAR.scaled | 6.99713258060345e−05 | 1.00007 |

The 6-cytokine panel approach was further used to predict the odds of having a ruptured intracranial aneurysm. Values of intercept, coefficient, p-value, and odds ratio were obtained in a model trained in 32 human subjects (Table 11, Table 12, and Equation 6) using covariates selected from univariable logistic regression.

TABLE 11
Values of odds ratio and p-value in model trained
in 32 human using covariates selected from univariable
or multivariable logistic regression.

| Variable | Coefficient | p-value |
|---|---|---|
| MCP-2.scaled | 0.933 | 1.000 |
| IL12-p40.scaled | 0.878 | 1.000 |
| sTNF-RI.scaled | 0.989 | 1.000 |
| MIG.scaled | 1.197 | 0.999 |
| IL-1ra.scaled | 1.291 | 1.000 |
| IL-1alpha.scaled | 0.811 | 1.000 |

TABLE 12
Values of intercept, coefficient, and odds ratio in model
derived trained in 32 human subjects using covariates
selected from univariable logistic regression.

| Coefficient | Value | Odds Ratio |
|---|---|---|
| Intercept | −53.78 | 0 |
| MCP-2.scaled | −0.07 | 0.93 |
| IL12-p40.scaled | −0.13 | 0.88 |
| sTNF-RI.scaled | −0.01 | 0.99 |
| MIG.scaled | −0.18 | 1.2 |
| IL-1ra.scaled | 0.26 | 1.3 |
| IL-1alpha.scaled | −0.21 | 0.81 |

To generate a prediction for odds of having a ruptured intracranial aneurysm, insert values of each variable from the experimental data and solve the following equation (Equation 6 and Table 12).

Equation 6. Specific Form of the Equation in Model Trained in 32 Human Subjects Using Covariates Selected from Univariable Logistic Regression:

$$P = \frac{{e}^{- 53.78 + ( - \text{.07} \times MCP - 2 ) + ( - 0.13 \times IL 12 - p 40 ) + ( - 0.01 \times sTNF - RI ) + ( 0.18 \times MIG ) + ( 0.26 \times IL - 1 ra ) + ( - 0.21 \times IL - alpha )}}{1 + {e}^{- 53.78 + ( - \text{.07} \times MCP - 2 ) + ( - 0.13 \times IL 12 - p 40 ) + ( - 0.01 \times sTNF - RI ) + ( 0.18 \times MIG ) + ( 0.26 \times IL - 1 ra ) + ( - 0.21 \times IL - alpha )}}$$

Application of subsequent cytokine panel tests were then used to predict and differentiate distinct inflammatory subgroups within peripheral blood samples from patients harboring known aneurysms (Tables 12-15).

TABLE 12
Application of a subsequent 6-cytokine panel test to predict
and differentiate between subgroup 2 and other groups within
the peripheral blood of patients harboring aneurysms.

| Variable | Coefficient | Odds Ratio |
|---|---|---|
| Intercept | −1.75140761743064 | 0.1735295 |
| GDNF.scaled | −0.0017619337048749 | 0.9982396 |
| IGFBP-4.scaled | −6.222445617901e−05 | 0.9999378 |
| IL-3.scaled | −0.00101979745558385 | 0.9989807 |
| SCF.scaled | 0.000898225329140785 | 1.0008986 |
| HGF.scaled | −0.000366712602235642 | 0.9996334 |
| IL17.scaled | 0.00226184265205335 | 1.0022644 |

TABLE 13
Application of a subsequent 4-cytokine panel test to predict
and differentiate between subgroup 3 and other groups within
the peripheral blood of patients harboring aneurysms.

| Variable | Coefficient | Odds Ratio |
|---|---|---|
| Intercept | −3.26737811233188 | 0.0381062 |
| Il-16.scaled | 0.000368881510240146 | 1.0003689 |
| dtks.scaled | 0.00037248416843843 | 1.0003726 |
| EGF-R.scaled | 0.00022026670822369 | 1.0002203 |
| MIP-3-beta.scaled | 0.00214396661621934 | 1.0021463 |

TABLE 14
Application of a subsequent 3-cytokine panel test to predict
and differentiate between subgroup 4 and other groups within
the peripheral blood of patients harboring aneurysms.

| Variable | Coefficient | Odds Ratio |
|---|---|---|
| Intercept | −3.42264202173536 | 0.0326261 |
| GCP-2.scaled | 0.00190671019582332 | 1.0019085 |
| LIGHT.scaled | 0.000530410055518138 | 1.0005306 |
| MCP-2.scaled | 0.00351569498953506 | 1.0035219 |

TABLE 15
Application of a subsequent 4-cytokine panel test to predict
and differentiate between subgroup 5 and other groups within
the peripheral blood of patients harboring aneurysms.

| Variable | Coefficient | Odds Ratio |
|---|---|---|
| Intercept | −1.36288785658345 | 0.2559206 |
| Fractalkine.scaled | −0.000571006897250064 | 0.9994292 |
| IGFBP-1.scaled | 0.000181132240743949 | 1.0001811 |
| bFGP.scaled | −0.000933951130906498 | 0.9990665 |
| IGF-ISR.scaled | −0.00236261931035077 | 0.9976402 |

### Example 5. Discussion

The present example summarizes and discusses the results described in Examples 1-4. The present disclosure shows a small cytokine panel can be used to detect human cerebral aneurysm formation and growth and guide clinical therapy. The present disclosure shows a simple, inexpensive blood test that allows for early detection and treatment. The present disclosure shows a cytokine panel that could be used to detect human cerebral aneurysm formation and growth and guide clinical therapy. Specifically, a whole blood-based, protein-based mini-cytokine panel works by detecting levels of cytokines and applying a set or sets of models to derive predictions, i.e., whether an aneurysm is present, the odds of an aneurysm rupture, and the inflammatory subgroup the aneurysm belongs to. Two six-cytokine panels of those on the array (I-309, IL-16, IL-1 alpha, MCP-2, MIP-1 delta, and uPAR; or I-309, IL-16, MCP-4, MIP-1 delta, CXCL7/NAP-2 and uPAR) would be used for a model to predict if a patient harbors an aneurysm. A five-cytokine panel (RANTES, IL-12 p40/p70, MIP-1α, sTNF.RI, and MCP-1) and a six-cytokine panel (MCP-2, IL12-p40, sTNF-RI, MIG<IL-1ra, and IL-1alpha) of those on the array are used for model 2 to predict if a patient with an aneurysm is at risk of rupture. Furthermore, patients harboring aneurysms can be further defined into subgroups based on subsequent cytokine panel test. The present disclosure shows that there are five distinct inflammatory subgroups of cerebral aneurysms and application of the models disclosed herein can differentiate between four of them via various additional smaller panels. Panels containing additional cytokines will continue to be further developed to account for different aneurysm subtypes given the dynamic aspect of inflammation in this disease. Identifying specific inflammatory subgroups will allow for a specific anti-inflammatory treatment approach.

There have been multiple different cytokines and inflammatory proteins reported to a have role in aneurysm formation. However, prior efforts to detect cerebral aneurysms have failed because: 1) it is unlikely a single biomarker is specific enough, since 2) there are multiple confounding variables such as age, sex, smoking status, presence of other vascular diseases, or underlying inflammatory state, and 3) samples of uncertain chronicity or timing of the disease process have resulted in mixed data sets. Some of the previous targets that have been suggested as potential biomarkers in cerebral aneurysms but have not been seriously pursued due to their non-specific nature include: complement C3c and c9, immunoglobulins (IgG, and IgM), CD68+ monocytes cells, M1 and M2 macrophages, mast cells, T and B lymphocytes, MPO, VCAM-1, CSF and ICAM-1(serum), CSF and E-selecting (serum), CSF-NfHSM135, and various serum molecules (e.g. VEGF, GM-CSF. IL-10, TNF-α, MCP-1, cortisol, T3 and free T4, elastase A1AT, LPA, and S-100).

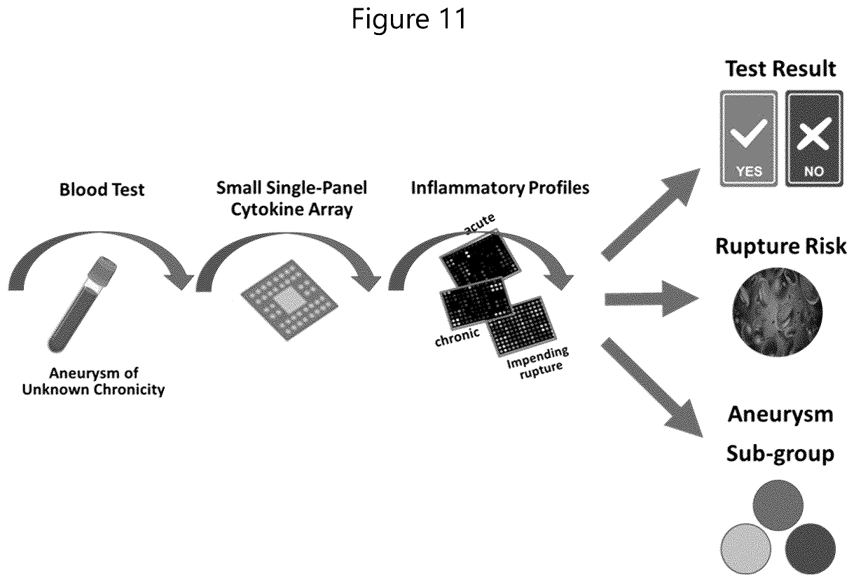

In summary, the present disclosure focused on defining inflammatory profiles for 1) a nascent aneurysm, 2) acute aneurysm, 3) fully developed aneurysm, 4) chronic aneurysm, and 5) impending rupture of the aneurysm. The present disclosure discovered a small cytokine panel capable of picking up aneurysm subtypes with very different inflammatory profiles. The basic idea behind the present disclosure is shown in the graphic in FIG. 11. The present disclosure first established murine inflammatory signature profiles (5 groups of n=3) (FIGS. 4A-4C), and then applied a LASSO method approach (blinded) to cytokine profiling in human patient samples. For analysis, human aneurysm dome was paired with STA tissue, and venous blood samples (blinded, n=21 for peripheral blood, n=3 for aneurysm dome, n=3 kit reference serum, total controls=11) (FIGS. 5A, 5C and 5D). The present disclosure discovered differential expression of inflammatory cytokines between patient peripheral blood and reference serum. The present disclosure used cytokine expression profiling to build models for both detecting cerebral aneurysms and predicting the odds of aneurysm rupture accurately in humans (FIG. 5E). Furthermore, the present disclosure found that the diagnostic test has application for defining particular aneurysms inflammatory subgroups (FIGS. 9A-9D), which demonstrates the diagnostic testing disclosed herein can be used in the development of treatment schemes (FIG. 14).

### Example 6. Material and Methods

The present example provides experimental procedures that were followed to complete studies disclosed in Examples 1-7.

Mouse intracranial aneurysm model. Murine intracranial aneurysms were created in female 8-12 week-old C57BL/6 mice (Charles River Laboratories, Wilmington, MA) following established protocols. Briefly, the left common carotid artery and the right renal artery are ligated to induce hypertension. One week later, an Alzet micro-osmotic pump model 1004 (DURECT Corp, Cupertino, CA) was implanted subdermally to deliver Angiotensin II (Bachem AG, Switzerland) at 1000 ng/kg/min; and 10 μL of 0.8% porcine elastase (Worthington Biochemical Corp, Lakewood, NJ) in normal saline was injected into the right basal cistern using stereotactic coordinates: 1.2 mm rostral of bregma, 0.7 mm lateral of midline and 5.3 mm ventral of the dorsal aspect of the skull. The animals were fed a hypertensive diet with 8% NaCl and 0.12% BAPN (TEKLAD). PBS-treated animals received 10 mL injection of phosphate buffered saline subcutaneously every two days. Control sham-surgery animals represented controls that had the incisions and surgical approach performed but no vessel ligation or intracranial injection. All animal experimentation was performed under the Institutional Animal Care and Use Committee-approved protocols of the University of Pittsburgh.

Mouse Carotid Aneurysm Model. Murine carotid aneurysms were created in female 8-12 week-old C57BL/6 mice (Charles River Laboratories, Wilmington, MA) using established protocols. Briefly, the right common carotid artery is exposed, ligated distally to create a stump, and exposed to 0.8% porcine elastase (Worthington Biochemical Corp, Lakewood, NJ) in normal saline over 20 minutes. The carotid aneurysms then develop over 3 weeks. Control sham-surgery animals represented controls that had the incisions and surgical approach performed but no vessel ligation.

Human aneurysm and artery specimens. Collection of human cerebral aneurysm and artery specimens was performed under the IRB-approved protocol and stored in patient de-identified THUNDA-Dome biobank (MMM). Patients gave written informed IRB research consent. All aneurysm and artery specimens were harvested from living patients at the time of craniotomy and aneurysm clipping surgery. Specimens were immediately placed in RNAlater stabilization solution (Invitrogen), flash frozen at −20° C., transferred to −80° C. for 24 hours, and then finally stored fresh-frozen in liquid nitrogen.

Mouse cytokine Arrays. Raybiotech cytokine arrays C1000 (AAM-CYT-1000, Raybiotech, Peachtree Corners, GA) were used to analyze 96 cytokines in aneurysm-induced cerebral vasculature at 2 and 3 weeks (n=3 each) and compared with reparixin-, clopidogrel-treated animals, and with sham surgery animals as controls (n=2).

Human cytokine Arrays. Raybiotech cytokine arrays C1000 (AAH-CYT-1000, Raybiotech, Peachtree Corners, GA) were used to analyze 120 cytokines in paired samples of peripheral venous blood from aneurysm patients undergoing clipping surgery (n=6), aneurysmal dome tissue (n=4), control superficial temporal arteries, or reference serum (n=3).

Statistical Analysis. Initial statistical analysis and raw data analysis was performed by the corresponding author of this application (KWN). De-identification of samples used for modeling and testing was then done (KWN). Statistical modeling and test building was performed in a blinded fashion.

Web- and mobile-based software. A R statistical package was used to design a web-based and/or mobile based application to properly assign risk stratification and need for surgery for patients with known cerebral aneurysms.

Although the presently disclosed subject matter and its advantages have been described in detail, it should be understood that various changes, substitutions, and alterations can be made herein without departing from the spirit and scope of the disclosure. Moreover, the scope of the present application is not intended to be limited to the particular embodiments of the process, machine, manufacture, and composition of matter, and methods described in the specification. As one of ordinary skill in the art will readily appreciate from the disclosure of the presently disclosed subject matter, processes, machines, manufacture, compositions of matter, or methods, presently existing or later to be developed that perform substantially the same function or achieve substantially the same result as the corresponding embodiments described herein can be utilized according to the presently disclosed subject matter. Accordingly, the appended claims are intended to include within their scope such processes, machines, manufacture, compositions of matter, or methods.

Various patents, patent applications, publications, product descriptions, protocols, and sequence accession numbers are cited throughout this application, the disclosure of which are incorporated herein by reference in their entireties for all purposes.

What is claimed is:

1. A method of treating an aneurysm in a subject in need thereof, comprising
a) measuring, in a biological sample of the subject, an expression level of (i) I-309, IL-16, IL-1 alpha, MCP-2, MIP-1 delta, and/or uPAR, (ii) I-309, IL-16, MCP-4, MIP-1 delta, CXCL7/NAP-2 and/or uPAR, or (iii) FasL and/or CCL22;
b) identifying the subject as having an aneurysm if the expression level of (i) I-309, IL-16, IL-1 alpha, MCP-2, MIP-1 delta, and/or uPAR, (ii) I-309, IL-16, MCP-4, MIP-1 delta, CXCL7/NAP-2 and/or uPAR, or (iii) FasL and/or CCL22 is increased relative to a first reference sample; and
c) administering an effective amount of an aneurysm inhibitor to the subject.

2. The method of claim 1, wherein the aneurysm inhibitor is a platelet inhibitor.

3. The method of claim 2, wherein the platelet inhibitor is selected from the group consisting of a glycoprotein IIB/IIIA inhibitor, a CXCL7 inhibitor, a CXCR1/2 inhibitor, and a combination thereof.

4. The method of claim 3, wherein the glycoprotein IIB/IIIA inhibitor is clopidogrel, a salt thereof, or a derivative thereof.

5. The method of claim 3, wherein the CXCL7 inhibitor is an antibody anti-CXCL7.

6. The method of claim 3, wherein the CXCR1/2 inhibitor is reparixin, a salt thereof, or a derivative thereof.

7. The method of claim 1, further comprising administering a therapeutically effective amount of a secondary aneurysm inhibitor.

8. A method for preventing or reducing the risk of growth and/or rupture of an aneurysm in a subject in need thereof, comprising:
a) measuring, in a biological sample of the subject, an expression level of (i) I-309, IL-16, IL-1 alpha, MCP-2, MIP-1 delta, and/or uPAR, (ii) I-309, IL-16, MCP-4, MIP-1 delta, CXCL7/NAP-2 and/or uPAR, or (iii) FasL and/or CCL22;
b) identifying the subject as having an aneurysm if the expression level of (i) I-309, IL-16, IL-1 alpha, MCP-2, MIP-1 delta, and/or uPAR, (ii) I-309, IL-16, MCP-4, MIP-1 delta, CXCL7/NAP-2 and/or uPAR, or (iii) FasL and/or CCL22 is increased relative to a first reference sample;
c) measuring, in the biological sample, an expression level of one or more cytokines;
d) determining whether the subject has or is at risk of rupture of the aneurysm; and
e) administering a therapeutically effective amount of an aneurysm inhibitor to the subject.

9. The method of claim 1, wherein the one or more cytokines are selected from the group consisting of RANTES, IL-12 p40/p70, MIP-1α, sTNF.RI, MCP-1, MCP-2, MCP-3, MIG, IL-1ra, IL1-α, or a combination thereof.

10. The method of claim 9, wherein
a) a reduced expression level of RANTES relative to a second reference sample indicates that the subject has or is at risk of rupture of the aneurysm;
b) a reduced expression level of IL-12 p40/p70 relative to the second reference sample indicates that the subject has or is at risk of rupture of the aneurysm;
c) a reduced expression level of MIP-1α relative to the second reference sample indicates that the subject has or is at risk of rupture of the aneurysm;
d) a reduced expression level of sTNF.RI relative to the second reference sample indicates that the subject has or is at risk of rupture of the aneurysm;
e) an increased expression level of MCP-1 relative to the second reference sample indicates that the subject has or is at risk of rupture of the aneurysm;
I) an increased expression level of MCP-2 relative to the second reference sample indicates that the subject has or is at risk of rupture of the aneurysm;
g) an increased expression level of MCP-3 relative to the second reference sample indicates that the subject has or is at risk of rupture of the aneurysm;
h) an increased expression level of MIG relative to the second reference sample indicates that the subject has or is at risk of rupture of the aneurysm;
i) an increased expression level of IL-1ra relative to the second reference sample indicates that the subject has or is at risk of rupture of the aneurysm; and/or
j) an increased expression level of IL1-α relative to the second reference sample indicates that the subject has or is at risk of rupture of the aneurysm.

11. The method of claim 1, wherein the aneurysm is a cerebral aneurysm.

12. The method of claim 1, wherein the biological sample is a blood sample, a serum sample, a plasma sample, or a cerebrospinal fluid sample.

13. The method of claim 1, wherein the biological sample is a blood sample.

14. The method of claim 1 further comprising monitoring the subject's responsiveness to an anti-aneurysm treatment, comprising:
a) measuring, in the biological sample, an expression level of one or more cytokines; and
b) determining whether the subject is responsive to the anti-aneurysm treatment;
wherein the subject is administered or has been administered with the anti-aneurysm treatment.

15. A method of identifying a subject having or at risk of developing an aneurysm by one or more computing systems, comprising:
receiving, from a client device via a user interface of a software executing on the client device, one or more inputs associated with the subject;
determining, based on one or more models, one or more measures regarding aneurysm presence and aneurysm rupture, wherein the one or more measures comprise one or more of a first probability of the subject harboring an aneurysm, a second probability of an ruptured aneurysm in the subject, or a third probability of an aneurysm with impending rupture in the subject; and
sending, to the client device via the user interface, instructions for presenting the one or more determined measures regarding aneurysm presence and aneurysm rupture.

16. The method of claim 15, wherein the one or more inputs comprise one or more of demographic information, a co-morbidity, an aneurysm size, an aneurysm location, or a cytokine.

17. The method of claim 15, wherein the one or more inputs comprise:
a) one or more cytokines selected from the group consisting of I-309, IL-16, IL-1 alpha, MCP-2, MIP-1 delta, uPAR or a combination thereof;
b) one or more cytokines selected from the group consisting of I-309, IL-16, MCP-4, MIP-1 delta, CXCL7/NAP-2, uPAR, or a combination thereof;
c) one or more cytokines selected from the group consisting of FasL, CCL22, or a combination thereof; or
d) one or more cytokines selected from the group consisting of RANTES, IL-12 p40/p70, MIP-1α, sTNF.RI, MCP-1, MCP-2, MCP-3, MIG, IL-1ra, IL1-α, or a combination thereof.

18. The method of claim 15, wherein the one or more models are generated based on one or more of retrospective human cytokine data or sample stratification based on t-SNE inflammatory cytokine analysis.

19. One or more computer-readable non-transitory storage media embodying software that is operable when executed to:
receive, from a client device via a user interface of a software executing on the client device, one or more inputs associated with the subject;
determine, based on one or more models, one or more measures regarding aneurysm presence and aneurysm rupture, wherein the one or more measures comprise one or more of a first probability of the subject harboring an aneurysm, a second probability of an ruptured aneurysm in the subject, or a third probability of an aneurysm with impending rupture in the subject; and
send, to the client device via the user interface, instructions for presenting the one or more determined measures regarding aneurysm presence and aneurysm rupture.

20. A system comprising: one or more processors; and a non-transitory memory coupled to the processors comprising instructions executable by the processors, the processors operable when executing the instructions to:
receive, from a client device via a user interface of a software executing on the client device, one or more inputs associated with the subject;
determine, based on one or more models, one or more measures regarding aneurysm presence and aneurysm rupture, wherein the one or more measures comprise one or more of a first probability of the subject harboring an aneurysm, a second probability of an ruptured aneurysm in the subject, or a third probability of an aneurysm with impending rupture in the subject; and
send, to the client device via the user interface, instructions for presenting the one or more determined measures regarding aneurysm presence and aneurysm rupture.
